(19) United States
(12) Patent Application Publication
(10) Pub. No.: US 2025/0269014 A1
(43) Pub. Date: Aug. 28, 2025

(54) POLYSACCHARIDES FOR USE IN TREATING SARS-COV-2 INFECTIONS

(71) Applicant: NDPD PHARMA, INC., Needham, MA (US)

(72) Inventor: David PLATT, Newton, MA (US)

(21) Appl. No.: 18/035,640

(22) PCT Filed: Nov. 5, 2021
(86) PCT No.: PCT/US2021/058310

§ 371 Date: May 5, 2023

Related U.S. Application Data
(60) Provisional application No. 63/110,736, filed on Nov. 6, 2020.

(51) Int. Cl. A61K 39/39; A61K 31/736; A61K 39/00; A61P 31/14

(52) U.S. Cl. CPC A61K 39/39; A61K 31/736; A61P 31/14; A61K 2039/542; A61K 2039/55583; A61K 2039/575; A61K 2039/58

(57) ABSTRACT

The invention provides a method for treating SARS-CoV-2 by administering an effective amount of galactomannans to a subject in need thereof.

### BACKGROUND OF THE INVENTION

SARS-CoV-2 is a single-stranded RNA virus that causes the disease COVID-19. The ongoing SARS-CoV-2 pandemic has caused more than 3,000,000 deaths. There are no known treatments that lower the infectivity of SARS-CoV-2. Additional treatments for SARS-CoV-2 infections that lower the infectivity of SARS-CoV-2 are needed.

### SUMMARY OF THE INVENTION

The present invention provides a method of treating a SARS-CoV-2 infection in a subject in need thereof, by administering to the subject an effective amount of galactomannans, or a pharmaceutical composition thereof.

In some embodiments of the method of the invention, administration of the effective amount of galactomannans, or a pharmaceutical composition thereof, reduces the SARS-CoV-2 infectivity of a subject relative to the SARS-CoV-2 infectivity of the subject in the absence of galactomannan administration.

In some embodiments of the method of the invention, administration of the effective amount of galactomannans, or a pharmaceutical composition thereof, decreases the copy number of SARS-CoV-2 RNA polynucleotides present in a biological sample from the subject, e.g., the nasal secretions, blood, saliva, sputum, serum, and/or stool, relative to the copy number of SARS-CoV-2 RNA polynucleotides present in a biological sample of the subject in the absence of galactomannan administration, e.g., prior to administration.

In some embodiments of the method of the invention, administration of the effective amount of galactomannans, or a pharmaceutical composition thereof, increases the cycle threshold (Ct) number of a SARS-CoV-2 gene, e.g., the envelope protein (E) gene, nucleocapsid (N) gene, and/or RNA-dependent RNA (Rd/Rp) polymerase gene, e.g., measured in a real time polymerase chain reaction (PCR) experiment, e.g., using a biological sample obtained from the subject, e.g., nasal secretions, blood, saliva, sputum, serum, and/or stool, relative to the Ct number of the gene in the absence of galactomannans administration, e.g., prior to administration.

In some embodiments, the galactomannans are formulated in a pharmaceutical composition with a pharmaceutically acceptable carrier. In some embodiments, the pharmaceutical composition is a solid oral dosage form, e.g., a chewable tablet. In some embodiments, including any of the foregoing embodiments, the subject holds the chewable tablet in their mouth for at least one minute before swallowing, e.g., at least one minute, at least two minutes, at least three minutes, at least four minutes, at least five minutes, or one to two minutes, one to three minutes, one to four minutes, or one to five minutes, before swallowing.

In some embodiments, the galactomannans, or pharmaceutical composition thereof, is administered to the subject at least one time per day, e.g., at least two times per day, at least three times per day, at least five times per day, at least six times per day, at least seven times per day, at least eight times per day, at least nine times per day, at least ten times per day, e.g., up to 10 times a day. In some embodiments, the galactomannans, or pharmaceutical composition thereof, is administered to the subject one time per hour during each hour that the subject is awake, e.g., up to 10 times a day.

In some embodiments, the galactomannans are administered to the subject at least thirty minutes after the subject has last eaten, e.g., at least one 60 minutes, at least 90 minutes, at least 120 minutes after the subject has last eaten.

In some embodiments, the galactomannans are from the plants, e.g., the seeds of, Trigonella foenum-graecum, Cyamopsis tetragonoloba, Acacia senegal, Acacia seyal, Ceratonia siliqua, Cassia fistula, and/or Caesalpinia spinosa.

In some embodiments, the infection is a symptomatic infection. In some embodiments, the infection is an asymptomatic infection. In some embodiments, the infection is an rRT-PCR positive infection. In some embodiments, the infection is a mild infection. In some embodiments, the infection is a moderate infection. In some embodiments, the infection is a severe infection.

In some embodiments, the method of the invention results in a higher Immunoglobulin G antibody titer in the subject relative to the Immunoglobulin G antibody titer observed in the absence of galactomannan administration, e.g., prior to administration.

In a second aspect, the present invention provides a method for reducing the number of SARS-CoV-2 virons in a sample of cells by contacting the sample with an effective amount of galactomannans.

In some embodiments, the number of virons in the sample of cells is reduced by at least 50%, e.g., at least 60%, at least 70%, at least 80%, at least 90%, at least 95%, at least 99%. In some embodiments, the sample of cells is substantially free of virons following being contacted with the effective amount of galactomannans.

In some embodiments, the sample of cells is exposed to SARS-CoV-2 virons prior to being contacted with the effective amount of galactomannans. In some embodiments, the cells are contacted with the effective amount of galactomannans prior to being exposed to SARS-CoV-2 virons.

Comparison of the change of levels or values effectuated by the methods of the invention, e.g., in the level of SARS-CoV-2 infectivity in a subject, in the copy number of SARS-CoV-2 RNA polynucleotides present in a biological sample, in the copy number of a SARS-CoV-2 gene, in the titer of Immunoglobulin G antibodies in a subject, or in a number of SARS-CoV-2 virons present in a sample, e.g., in a sample of cells, may be made relative to any appropriate control, e.g., relative to a pre-determined level or value.

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the term “approximately” may be understood to permit standard variation as would be understood by those of ordinary skill in the art; and (v) where ranges are provided, endpoints are included.

As used herein, the term “about” represents a value that is in the range of ±10% of the value that follows the term “about.” Reference to “about” a value or parameter herein includes (and describes) variations that are directed to that value or parameter per se. For example, description referring to “about X” includes description of “X”.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, and vitreal.

As used herein, the term “biological sample” is a sample obtained from a subject including but not limited to blood (e.g., whole blood, processed whole blood (e.g., a crude whole blood lysate), serum, plasma, and other blood derivatives), bloody fluids (e.g., wound exudate, phlegm, bile, and the like), cerebrospinal fluid (CSF), urine, synovial fluid, breast milk, sweat, tears, saliva, semen, feces, vaginal fluid or tissue, sputum (e.g., purulent sputum and bloody sputum), nasopharyngeal aspirate or swab, lacrimal fluid, mucous, or epithelial swab (buccal swab), tissues (e.g., tissue biopsies (e.g., skin biopsies (e.g., from wounds, burns, or tick bites), muscle biopsies, or lymph node biopsies)), including tissue homogenates), organs, bones, teeth, among others. In some embodiments, the biological sample contains cells and/or cell debris derived from the subject from which the sample was obtained. In some embodiments, the subject is a host of a pathogen, e.g., SARS-CoV-2, and the biological sample obtained from the subject includes subject (host)-derived cells and/or cell debris, as well as one or more pathogen cells or viral particles, e.g., SARS-CoV-2 viral particles. In some embodiments, the biological sample contains nucleic acids, e.g., DNA and/or RNA, derived from the subject from which the sample was obtained, as well as nucleic acids derived from the pathogen cells or viral particles, e.g., nucleic acids derived from SARS-CoV-2.

A “symptomatic infection” indicates the subject infected with SARS-CoV-2 has one or more symptoms of SARS-CoV-2 infection including, but not limited to: fever, chills, cough, shortness of breath or difficulty breathing, fatigue, muscle or body aches, confusion, headache, inability to wake up or stay awake, pale, gray, or blue-colored skin, lips, or nail beds, loss of taste or smell, sore throat, congestion or runny nose, nausea, vomiting, or diarrhea.

An “asymptomatic infection” indicates the subject infected with SARS-CoV-2 has not developed any symptoms of SARS-CoV-2 infection. An asymptomatic infection includes both subjects who later go on to develop one or more symptoms, and subjects who never develop one or more symptoms.

A “rRT-PCR positive infection” indicates that the subject infected with SARS-CoV-2 has developed a sufficient viral load for viral RNA polynucleotides to be detected by a real-time reverse transcription polymerase chain reaction test.

A “mild infection” indicates that the subject infected with SARS-CoV-2 experiences symptoms including a fever equal to or below about 100 degrees Fahrenheit, a cough, chills, fatigue, muscle or body aches, confusion, headache, loss of taste or smell, sore throat, congestion, runny nose, nausea, vomiting, or diarrhea.

A “moderate infection” indicates that the subject infected with SARS-CoV-2 experiences symptoms including a fever above about 100 degrees Fahrenheit, some shortness of breath, or chills with repeated shaking, in addition to any of the symptoms experienced by a subject experiencing a mild SARS-CoV-2 infection.

A “severe infection” indicates that the subject infected with SARS-CoV-2 experiences symptoms including shortness of breath or difficulty breathing; inability to wake up or stay awake; pale, gray, or blue-colored skin, lips or nail beds; or persistent pain and pressure in chest; in addition to any of the symptoms experienced by a subject experiencing a mild or moderate SARS-CoV-2 infection.

As used herein, the Cycle threshold (Ct number) is the number of PCR cycles required for the fluorescent signal caused by the production of a particular RT-PCR product to exceed a predetermined threshold.

An “effective amount” of a compound may vary according to factors such as the disease state, age, sex, and weight of the individual, and the ability of the compound to elicit the desired response. A therapeutically effective amount encompasses an amount in which any toxic or detrimental effects of the compound are outweighed by the therapeutically beneficial effects. An effective amount also encompasses an amount sufficient to confer benefit, e.g., clinical benefit.

As used herein, “fractionation” refers to a process by which one or more components of a complex mixture of biomatter are separated from the remaining mixture.

As used herein, “infectivity” refers to the ability of an individual infected with SARS-CoV-2 to transmit the infection to another individual. The infectivity of an individual is determined by the level of SARS-CoV-2 virons in the individual, as measured by the Ct values expressed for the SARS-CoV-2 RNA polymerase (Rd/RP) gene, nucleocapsid (N) gene, and/or envelope (E) gene in a sample (e.g., a nasopharyngeal swab, a saliva sample, a stool sample, a serum sample, a blood sample) obtained from the patient.

The term “pharmaceutical composition,” as used herein, represents a composition containing a compound described herein formulated with a pharmaceutically acceptable excipient. Pharmaceutical compositions can be formulated, for example, for oral administration in unit dosage form (e.g., a tablet, capsule, caplet, gel cap, suspension, solution, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

As used herein, the term “subject” or “participant” or “patient” refers to any organism to which a compound or composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean both therapeutic treatment and prophylactic or preventive measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the subject; or enhancement or improvement of condition, disorder, or disease. Treatment includes eliciting a clinically significant response without excessive levels of side effects. Treatment also includes prolonging survival as compared to expected survival if not receiving treatment.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF THE DRAWINGS

FIG. 1 shows the structure of galactomannans employed in the present invention.

FIG. 2 shows the structure of galactomannans employed in the present invention.

FIG. 3 shows the structure of galactomannans employed in the present invention.

FIG. 4 shows the structure of galactomannans employed in the present invention.

FIG. 5 is a chart showing Ct values for the SARS-CoV-2 envelope gene in individuals treated with the galactomannans compared to Ct values for the SARS-CoV-2 envelope gene in individuals not treated with the galactomannans (control).

FIG. 6 is a chart showing Ct values for the SARS-CoV-2 RNA-dependent RNA polymerase gene and envelope gene in individuals treated with galactomannans compared to Ct values for the SARS-CoV-2 RNA-dependent RNA polymerase gene and envelope gene in individuals not treated with galactomannans (control).

FIG. 7 is a flow chart showing an experimental protocol used to measure the effect of galactomannans on the viral load of SARS-CoV-2 in Vero cells (ATCC@ CCL-81™).

FIG. 8 is a chart showing reduction in viral particles present in a sample of Vero cells (ATCC® CCL-81™) following administration of galactomannans.

FIG. 9 is a chart showing reduction in viral particles present in a sample of Vero cells (ATCC® CCL-81™) following administration of galactomannans.

FIG. 10 is a total ion chromatogram showing the results of gas chromatography/mass spectrometry analysis of partially methylated alditol acetate derivatives made from a sample of galactomannans.

FIG. 11 is a ¹H-NMR spectrum of the anomeric region of galactomannans.

FIG. 12 is a full range ¹H-NMR spectrum of galactomannans.

FIG. 13 is a HSQC spectrum of the anomeric region of galactomannans.

FIG. 14 is a HSQC spectrum of the glycosyl ring H/C region of galactomannans.

FIG. 15 is a TOCSY spectrum of galactomannans.

FIG. 16 is a NOESY spectrum of galactomannans.

### DETAILED DESCRIPTION

There are no approved therapeutics which limit or decrease the infectivity of SARS-CoV-2. The present inventors have discovered that galactomannans are an effective treatment for SARS-CoV-2. The present inventors have further discovered that galactomannans are effective at decreasing the infectivity of SARS-CoV-2. Therefore, one object of this invention is to provide a method for treating SARS-CoV-2 by administering an effective amount of galactomannans to a subject in need thereof. A second of object of this invention is to provide a method for treating SARS-CoV-2 that limits the infectivity of SARS-CoV-2, by administering an effective amount of galactomannans to a subject in need thereof. A third object of this invention is to provide a method for decreasing the number of SARS-CoV-2 virons in a sample of cells, by contacting the sample with an effective amount of galactomannans.

### SARS-CoV-2

SARS-CoV-2 is a single-stranded RNA virus that causes the disease COVID-19. Nonlimiting examples of symptoms of COVID-19 infections include fever, cough, headache, fatigue, breathing difficulties, nasal congestion and runny nose, sore throat, diarrhea, and loss of smell and taste. The majority of individuals who suffer from COVID infections experience mild or moderate symptoms. However, approximately 15% of individuals who become infected with SARS-CoV-2 experience severe symptoms. Nonlimiting examples of severe symptoms include dyspnea, hypoxia, respiratory failure, shock, multiorgan dysfunction, or death. A subset of patients who become infected with SARS-CoV-2 experience “long-haul infections” in which COVID-19 symptoms including but not limited to fatigue, headaches, shortness of breath, loss of smell, muscle weakness, low fever, and cognitive dysfunction continue for a period of time (e.g., days, weeks, months) following their diagnosis.

COVID-19 transmission is thought to occur mainly through respiratory route via SARS-CoV-2 virions which are contained in the respiratory droplets and/or aerosols of individuals infected with COVID-19. Transmission occurs when the respiratory droplets or aerosols enter the mouth, nose, or eyes of a second individual. Approximately 1,000 COVID virons are believed to be sufficient to initiate a new infection.

### Detection of SARS-CoV-2 in Biological Samples

SARS-CoV-2 infections are commonly diagnosed by detection of viral nucleic acids via real-time reverse transcription polymerase chain reaction (rRT-PCR). Samples for the diagnosis of a COVID-19 infection may be biological samples obtained from a subject, e.g., nasal secretions, (e.g., material obtained from the nasal passages and/or sinuses, optionally via nasopharyngeal swab), or blood, saliva, sputum, stool, or serum of a subject.

PCR experiments involve repeated cycles of heating and cooling a buffered mixture, e.g., of nucleotide triphosphates, a polynucleotide from a sample of interest, a probe polynucleotide with a sequence complementary to a portion of the nucleotide sequence in the sample that is intended to be amplified (the target sequence), and enzymes capable of catalyzing the chemical reactions that lead to the amplification of the sequence. In rRT-PCR experiments, the progress of polynucleotide amplification reactions is indirectly measured by observing changes in fluorescence that occur upon binding of fluorophore-containing probe nucleotides to nucleotides containing the target sequence. Target sequences present in high concentrations in a sample analyzed by rRT-PCR will have lower Ct values than target sequences present in low concentrations in rRT-PCR samples.

Multiple nucleotide molecules produced by SARS-CoV-2 may be detected using rRT-PCR. Nonlimiting examples of nucleotide molecules produced by SARS-CoV-2 that may be detected via rRT-PCR include the portion of the SARS-CoV-2 RNA genome encoding the envelope protein (the E gene) the portion of the SARS-CoV-2 RNA genome encoding the nucleocapsid protein (the N gene), and the RdRp gene, the portion of the SARS-CoV-2 RNA genome encoding the RNA dependent RNA polymerase protein (the Rd/RP gene), may be detected using rRT-PCR. In some embodiments, the E gene, N gene, and Rd/Rp gene are detected individually. In some embodiments, multiple genes are detected in a single rRT-PCR experiment (e.g., the Rd/Rp gene+the N gene).

The Ct value measured for a given gene in a biological sample is related to the copy number of the gene (i.e., the number of copies of the gene present in the sample). The copy number of the SARS-CoV-2 E gene, N gene, and/or Rd/RP gene in a specimen (e.g., a sample of nasal secretions, a blood sample, a stool sample, a saliva sample, a sputum sample, a serum sample, the lysate of a sample of cells, a sample of cell supernatant) may be calculated from the Ct value of the gene in the sample.

The infectivity of a subject infected with SARS-CoV-2 is related to the quantity of SARS-CoV-2 virons present in the subject (e.g., the viral load of the subject). The copy number of SARS-CoV-2 E gene, N gene, and/or Rd/RP gene in a sample (e.g., a sample of nasal secretions, a blood sample, a stool sample, a saliva sample, a sputum sample, a serum sample) obtained from a subject infected with SARS-CoV-2 is related to the number of SARS-CoV-2 virons present in the subject. A subject with a higher number of SARS-CoV-2 virons in their body (e.g., in their nasal secretions, blood, stool, saliva, sputum, serum) is likelier to transmit a SARS-CoV-2 infection than a subject with a lower number of SARS-CoV-2 virons in their body. Thus, the infectivity of a subject infected with SARS-CoV-2 can be measured via the Ct value of the SARS-CoV-2 E gene, N gene, and/or RdRp gene measured in a sample (e.g., a sample of nasal secretions, a blood sample, a stool sample, a saliva sample, a sputum sample, a serum sample) obtained from the subject.

### Galactomannans

Galactomannans are polysaccharides derived from plant biomass containing mannose or galactose moieties, or both groups, as the main structural components. The galactomannans described herein are a mixture of complex carbohydrates and include (1-6)-alpha-D-mannopyranosyl, 4-linked mannopyranosyl residues, 6-linked mannopyranosyl residues, 4-linked galactopyranosyl residues, 6-linked galactopyranosyl residues, 4-linked glucopyranosyl residues, 6-linked glucopyranosyl residues, 4,6-linked mannopyranosyl residues, 4,6-linked glucopyranosyl residues, terminal mannopyranosyl residues, terminal glucopyranosyl residues, and/or terminal galactopyranosyl residues. In some embodiments, the galactomannans described herein include linear chains of (1-4)-beta-D-mannopyranosyl units with alpha-D-galactopyranosyl units attached by 1-6 linkages. The carbohydrates may be in the range of 500-1000 D, 10 kD to 50 kD (e.g., 20 kD-40 kD), and/or 50-500 kD. In preferred embodiments, the galactomannans are water soluble.

Exemplary sources of galactomannans are one or more of Trigonella foenum-graecum, Cyamopsis tetragonoloba, Acacia senegal, Acacia seyal, Ceratonia siliqua, Cassia fistula, Senna obtusifolia, Senna tora, and Caesalpinia spinosa. In some embodiments, the galactomannans are one or more of fenugreek (e.g., from Trigonella foenum-graecum) galactomannans; Guar (e.g., from Cyamopsis tetragonoloba) galactomannans; Tara (e.g., from Caesalpinia spinosa or Tara spinosa) galactomannans; locust bean gum (e.g., from Ceratonia siliqua) galactomannans; and cassia gum (e.g., from Senna obtusifolia or Senna tora) galactomannans. In some embodiments, the galactomannans include gum acacia (e.g., from Acacia Senegal or Acacia seyal).

In some embodiments, the galactomannans are a mixture of any combination of fenugreek galactomannans, guar galactomannans, tara galactomannans, locust bean gum galactomannans, and cassia gum galactomannans. An exemplary galactomannan is Prolectin-M, as described herein.

In some embodiments, the galactomannans are chemically modified. For example, hydroxyethyl, hydroxypropyl and carboxymethylhydroxypropyl substitutions may be made to the galactomannans of the present invention. Non-ionic modifications to the galactomannans, such as those containing alkoxy and alkyl (C1-C6) groups, may be made to the galactomannans of the present invention. Anionic substitution may also be made to the galactomannans of the present invention.

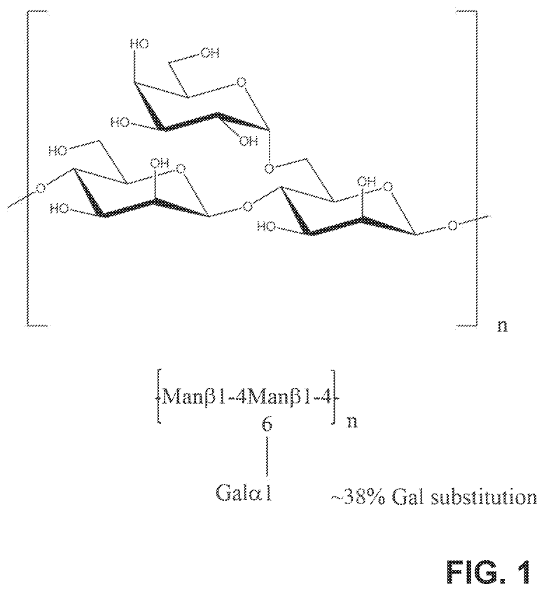

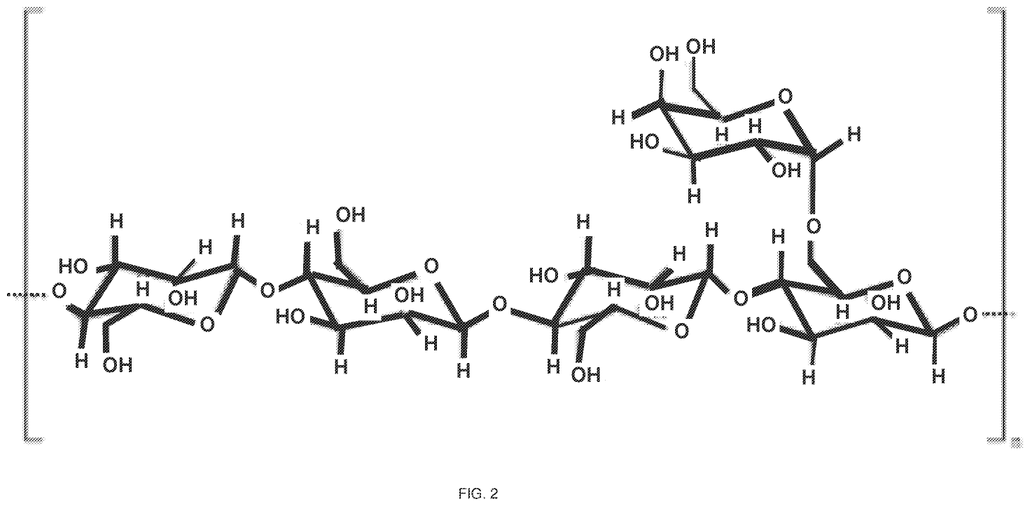

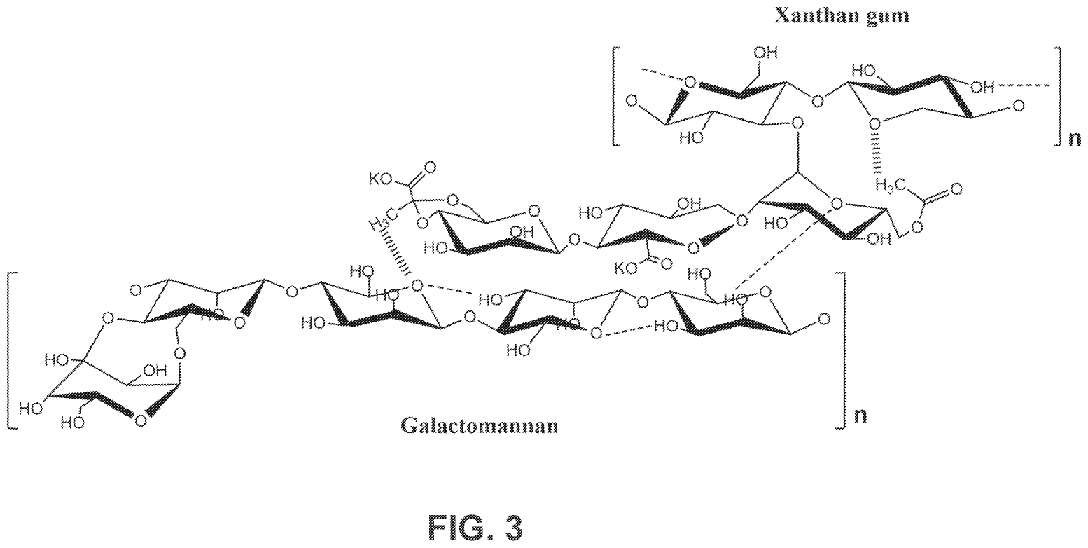

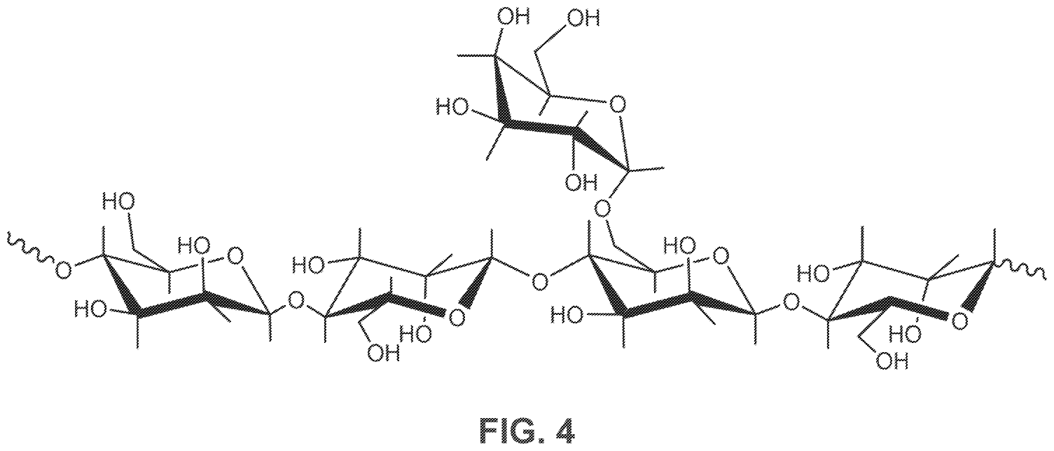

In some embodiments, galactomannans of the present invention have the structures shown in FIGS. 1-4.

In some embodiments, the galactomannans include at least one polysaccharide of high molecular weight and at least one polysaccharide of low molecular weight. In some embodiments, galactomannans include at least one polysaccharide of high molecular weight, at least one polysaccharide of low molecular weight, and at least one oligosaccharide, monosaccharide, and/or sugar alcohol.

In some embodiments, the polysaccharide of low molecular weight has a molecular weight of about 5-50 kD, e.g., about 10-40 kD, about 15-35 kD, or about 20-30 kD. In some embodiments, the polysaccharide of high molecular weight has a molecular weight of about 20-300 kD, e.g., about 25-200 kD, about 35-150 kD, or about 50-100 kD.

The one or more oligosaccharides, monosaccharides, and/or sugar alcohols may include, but are not limited to, galacturonic acid, galactose, mannose, mannitol, erythritol, sorbitol, inositol, raffinose (a nonreducing trisaccharide), galactinol (dulcitol), stachyose, verbascose, manninotriose, and higher homologs. In some embodiments, the oligosaccharides, monosaccharides, and/or sugar alcohols have a molecular weight of approximately 500-1,000 D, e.g., about 600-800 D, or about 650-700 D.

In some embodiments, the galactomannans include about 1 part of the at least one polysaccharide of high molecular weight, about 2 parts of the at least one purified mannan polysaccharide of low molecular weight, and about 1 part of oligosaccharides, monosaccharides, and/or sugar alcohol.

In some embodiments, the composition includes about 1% to about 50% (wt/wt) or about 1% to about 25% (wt/wt) of a polysaccharide of high molecular weight. In some embodiments, the composition includes about 20% to about 80% (wt/wt) of a polysaccharide of low molecular weight. In some embodiments, the composition includes about 40% to about 60% (wt/wt) of an oligosaccharide and/or monosaccharide. In some embodiments, the polysaccharide of high molecular weight has a high viscosity. In some embodiments, the polysaccharide of low molecular weight has a high solubility. In some embodiments, the ratio of low molecular weight polysaccharide to high molecular weight polysaccharide may be about 2 to 1 (wt/wt), 20 to 1 (wt/wt), and up to about 100 to 1 (wt/wt), inclusive of all ranges and sub-ranges in between. In some embodiments, the compositions described above may optionally include one or more additional additives. In an illustrative embodiment, an additional additive may include one or more sugar alcohols, including, but not limited to, sorbitol, erithritol, inositol, and other sugar alcohols of the type. A non-limiting list of other potential additional additives includes vitamins and minerals at their recommended % daily value requirements (for example, see www.USDA.gov).

In some embodiments, the galactomannans of the present invention bind to SARS-CoV-2 virons by a specified amount. Contacting a sample of SARS-CoV-2 virons, e.g., a sample of SARS-CoV-2 virons in a sample of cells (e.g., Vero (African green monkey kidney cells, Vero (ATTC® CCL-81™))) or a sample of SARS-CoV-2 virons in a biological sample, e.g., a biological sample obtained from a subject infected with SARS-CoV-2, with the galactomannans of the invention results in a decrease in the quantity of SARS-CoV-2 virons in the sample, e.g., as determined by a rRT-PCR experiment. Contacting the sample of SARS-CoV-2 virons with the galactomannans of the invention results in at least an about 80% reduction in the quantity of SARS-CoV-2 virons, e.g., an about 80% reduction an about 81% reduction, an about 82% reduction, an about 83% reduction, an about 84% reduction, an about 85% reduction, an about 86% reduction, an about 87% reduction, an about 88% reduction, an about 89% reduction, an about 90% reduction, an about 91% reduction, an about 92% reduction, an about 93% reduction, an about 94% reduction, an about 95% reduction, an about 96% reduction, an about 97% reduction, an about 98% reduction, an about 99% reduction, an about 100% reduction of the quantity of SARS-CoV-2 virons in the sample of SARS-CoV-2 virons, e.g., as determined by a rRT-PCT experiment.

The galactomannans may vary in the composition of its constituent carbohydrates. In some embodiments, the constituent carbohydrates vary in the ratio of galactose to mannose. Specifically, they may include about 95% galactose and about 5% mannose, about 90% galactose and about 10% mannose, about 80% galactose and about 20% mannose, about 70% galactose and about 30% mannose, about 60% galactose and about 40% mannose, about 50% galactose and about 50% mannose, about 40% galactose and about 60% mannose, about 30% galactose and about 70% mannose, about 20% galactose and about 80% mannose, about 10% galactose and about 90% mannose, less than about 5% galactose and greater than about 95% mannose, greater than 95% galactose and less than 5% mannose, greater than 90% galactose and less than 10% mannose, greater than 80% galactose and less than 20% mannose, greater than 70% galactose and less than 30% mannose, greater than 60% galactose and less than 40% mannose, greater than 50% galactose and less than 50% mannose, greater than 40% galactose and less than 60% mannose, greater than 30% galactose and less than 70% mannose, greater than 20% galactose and less than 80% mannose, greater than 10% galactose and less than 90% mannose, greater than 5% galactose and less than 95% mannose, 95%±5% galactose and 5%±0.5% mannose, 90%±9% galactose and 10%±1% mannose, 80%±8% galactose and 20%±2% mannose, 70%±7% galactose and 30%±3% mannose, 60%±6% galactose and 40%±4% mannose, 50%±5% galactose and 50%±5% mannose, 40%±4% galactose and 60%±6% mannose, 30%±3% galactose and 70%±7% mannose, 20%±2% galactose and 80%±8% mannose, 10%±1% galactose and 90%±9% mannose, less than 5%±0.5% galactose and greater than 95%±5% mannose.

The following description of galactomannan activity is provided without wishing to be bound by theory. The N-terminal domain (NTD) of the SARS-CoV-2 spike protein is essential for vial entry into human cells. Portions of the SARS-CoV-2 spike protein resemble human galectins, which contain a highly conserved carbohydrate-binding domain that binds a variety of galactose-containing saccharides. In some embodiments, the galactomannans bind to the NTD of the SARS-CoV-2 spike protein. In some embodiments, binding of galactomannans to the NTD of the SARS-CoV-2 spike protein is deleterious to the ability of SARS-CoV-2 to enter a human cell.

In some embodiments, galactomannans bind to galectins on the surface of the SARS-CoV-2 viral particle that are not the NTD of the SARS-CoV-2 spike protein. In some embodiments, galactomannans prevent entry of SARS-CoV-2 virons into human cells by allosterically inhibiting galectins on the surface of the SARS-CoV-2 envelope. In some embodiments, galactomannans bind to galectins on the surface of human cells. In some embodiments, the binding of galactomannans to galectins on the surface of human cells is deleterious to the ability of SARS-CoV-2 to enter human cells. In some embodiments, galactomannans bind to carbohydrates that are displayed on the surface of the SARS-CoV-2 viral particle. In some embodiments, the binding of galactomannans to carbohydrates displayed on the surface of the SARS-CoV-2 viral particle is deleterious to the ability of SARS-CoV-2 to enter human cells. In some embodiments, galactomannans recruit elements of the immune system, (e.g., leukocytes) to SARS-CoV-2 particles. In some embodiments, galactomannans deactivate SARS-CoV-2 particles, which are then eliminated by the liver.

In some embodiments, galactomannans stimulate the immune response against SARS-CoV-2 in a subject. One element of the immune response of production of immunoglobulin G (IgG). In some embodiments, administration of galactomannans results in a higher IgG antibody titer in the subject relative to the IgG antibody titer observed in the absence of galactomannan administration.

In some embodiments, galactomannans bind to galectins on viruses other than SARS-CoV-2. In some embodiments, galactomannans prevent the entry of viruses other than SARS-CoV-2, e.g., adenoviruses or retroviruses, into human cells by allosterically inhibiting galectins on the surface of the viruses.

In some embodiments, the binding of galactomannans to galectins on the surface of human cells is deleterious for the ability of viruses other than SARS-CoV-2 to enter human cells.

In some embodiments, the galactomannans bind to carbohydrates that are displayed on the surface of viruses other than SARS-CoV-2 (e.g., adenoviruses or retroviruses). In some embodiments, the binding of the carbohydrates is deleterious to the ability of the viruses to enter human cells.

In some embodiments, the galactomannans recruit elements of the immune system, (e.g., leukocytes) to viral particles other than SARS-CoV-2 particles. In some embodiments, the galactomannans deactivate the viral particles, which are then eliminated by the liver.

### Pharmaceutical Compositions

The administration of the galactomannans may be by any suitable means that results in treatment of a SARS-CoV-2 infection. The galactomannans may be contained in any appropriate amount in any suitable carrier substance and is generally present in an amount of 1-95% by weight of the total weight of the composition. The composition may be provided in a dosage form that is suitable for the sublingual, buccal, oral, parenteral (e.g., intravenously, intramuscularly), pulmonary, intranasal, transdermal, vaginal, or rectal administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, sprays, vapors, or aerosols. The pharmaceutical compositions may be formulated according to conventional pharmaceutical practice (see, e.g., Remington: The Science and Practice of Pharmacy, (23rd ed.) ed. A. Adejare, 2020, Academic Press, Philadelphia, PA).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the active compound within the body over an extended period of time; (ii) formulations that after a predetermined lag time create a substantially constant concentration of the active compound within the body over an extended period of time; and (iii) formulations that sustain active compound action during a predetermined time period by maintaining a relatively, constant, effective active compound level in the body with concomitant minimization of undesirable side effects associated with fluctuations in the plasma level of the active compound (sawtooth kinetic pattern).

Administration of the galactomannans in the form of a controlled release formulation is especially preferred in cases in which the active compound, either alone or in combination with a second agent, at therapeutic levels produces unwanted side effects, such as nausea.

Any one of a number of strategies can be pursued to obtain controlled release of the active compound. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the active compound in a controlled manner. Examples include single or multiple unit tablet or capsule compositions, oil solutions, suspensions, emulsions, microcapsules, microspheres, nanoparticles, patches, and liposomes.

### Solid Dosage Forms for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These compositions can be prepared in a variety of ways well known in the pharmaceutical art and can be made so as to release the galactomannans in specific segments of the gastrointestinal tract at controlled times by a variety of excipients and formulation technologies. For example, formulations may be tailored to address a specific disease, to achieve plasma levels of the galactomannans required to achieve therapeutic efficacy, to enable a desired duration of drug effect, and to provide a set of compositions with varying drug release.

The oral dosage forms contemplated by the invention may include the galactomannans in a mixture with non-toxic pharmaceutically acceptable excipients. Pharmaceutically acceptable excipients are known to the skilled artisan. Excipients may be, for example, inert diluents or fillers such as sucrose, sorbitol, sugar, mannitol, microcrystalline cellulose, in particular microcrystalline cellulose PH101 or microcrystalline cellulose PH200, starches including potato starch, calcium carbonate, sodium chloride, lactose, calcium phosphate, calcium sulfate, or sodium phosphate; disintegrants such as crospovidone, sodium alginate, colloidal magnesium aluminum silicate, calcium silicate, sodium starch glycolate, acrylic acid derivatives, microcrystalline cellulose, sodium carboxymethyl cellulose, calcium carboxymethyl cellulose, modified cellulose gum, cross-linked povidone, alginic acid and alginates, pregelatinised starch, modified corn starch cellulose derivatives including microcrystalline cellulose, starches including potato starch, croscarmellose sodium, alginates, or alginic acid; binders such as sucrose, glucose, sorbitol, acacia, alginic acid, gelatin, starch, pregelatinized starch, microcrystalline cellulose, magnesium aluminum silicate, carboxymethylcellulose sodium, methylcellulose, hydroxypropyl methylcellulose, hydroxypropyl cellulose EXF, ethylcellulose, polyvinylpyrrolidone, or polyethylene glycol; lubricants and/or glidants such as colloidal silicon dioxide, particularly colloidal silicon dioxide Cab-O-Sil® M5P, glycerol tribehenate, magnesium stearate, calcium stearate, talc, sodium stearyl fumarate, sodium behenate, stearic acid, cetyl alcohol, polyoxyethylene glycol, leucine, sodium benzoate, stearates, polyethylene glycol, glyceryl monostearate, glyceryl palmitostearate, liquid paraffin, poloxamer, sodium lauryl sulphate, magnesium lauryl sulphate, hydrogenated castor colloidal silicon dioxide, palmitostearate, stearic acid, zinc stearate, stearyl alcohol, silicas, or hydrogenated vegetable oil; anti-caking agents such as colloidal silicon dioxide, microcrystalline cellulose, tricalcium phosphate, microcrystalline cellulose, magnesium stearate, sodium bicarbonate, sodium ferrocyanide, potassium ferrocyanide, calcium ferrocyanide, calcium phosphate, sodium silicate, colloidal silicon dioxide, calcium silicate, magnesium trisilicate, talcum powder, sodium aluminosilicate, potassium aluminum silicate, calcium aluminosilicate, bentonite, aluminum silicate, stearic acid, polydimethylsiloxane. Other pharmaceutically acceptable excipients may be colorants, flavoring agents, plasticizers, humectants, and buffering agents.

Suitable pharmaceutical carriers, as well as pharmaceutical necessities for use in pharmaceutical formulations, are described in Remington: Remington: The Science and Practice of Pharmacy, (23rd ed.) ed. A. Adejare., 2020, Academic Press, Philadelphia, PA, and in the USP44/NF39 (United States Pharmacopeia and the National Formulary) or corresponding European or Japanese reference documents.

The solid compositions of the invention may include a coating adapted to protect the composition from unwanted chemical changes, (e.g., chemical degradation prior to the release of the active substances). The coating may be applied on the solid dosage form in a similar manner as that described in Encyclopedia of Pharmaceutical Technology (4th ed.) ed. J. Swarbrick, 2013, CRC Press, Boca Raton, FL.

Powders and granulates may be prepared using the ingredients mentioned above in a conventional manner using, e.g., a mixer, a fluid bed apparatus, melt congeal apparatus, rotor granulator, extrusion/spheronizer, or spray drying equipment.

### Solid Oral Dosage Form

In some embodiments, the pharmaceutical composition of the galactomannans is formulated in a solid oral dosage form. In some embodiments, the solid oral dosage form of the galactomannans is intended to be dissolved in the mouth of the subject. In some embodiments, the solid oral dosage form is chewable. In some embodiments, the subject chews the solid oral dosage form and holds the solid oral dosage form of the galactomannans in their mouth for at least 1 minute, e.g., at least 2 minutes, at least 3 minutes, at least 4 minutes, at least 5 minutes prior to swallowing. In some embodiments, the subject does not chew the solid oral dosage form and holds the pharmaceutical composition of the galactomannans in their mouth for at least 1 minute, e.g., at least 2 minutes, at least 3 minutes, at least 4 minutes, at least 5 minutes prior to swallowing. In some embodiments, the subject holds the pharmaceutical composition of the galactomannans in their mouth until it is substantially dissolved. In some embodiments, the subject holds the composition of the galactomannans in their mouth until the galactomannans have contacted at least 50% (at least 50%, at least 60%, at least 70%, at least 80%, at least 90%, at least 95%, of the area of their oral mucosa.

### Dosages

The dosage of the composition used in the methods described herein can vary depending on many factors, e.g., the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The composition used in the methods described herein may be administered initially in a suitable dosage that may be adjusted as required, depending on the clinical response. In general, a suitable daily dose of a compound of the invention will be that amount of the compound that is the lowest dose effective to produce a therapeutic effect. Such an effective dose will generally depend upon the factors described above.

While the attending physician ultimately will decide the appropriate amount and dosage regiment, an effective amount may be between about 1 g of the galactomannans per day and about 60 g the galactomannans per day, e.g., between about 5 g/day and about 50 g/day, between about 10 g/day and about 40 g/day, between about 15 g/day and about 30 g/day. In some embodiments, an effective amount of the galactomannans may be between 1±0.1 g/day and 60±6 g/day, e.g., 5±0.5 g/day and 50±5 g/day, between about 10±1 g/day and 40±4 g/day, between about 15±1.5 g/day and 30±3 g/day. In some embodiments, an effective amount of the galactomannans may be between 1 g/day and 60 g/day, between about 5 g/day and about 50 g/day, between about 10 g/day and about 40 g/day, between about 15 g/day and about 30 g/day.

In some embodiments, an effective amount of the galactomannans may be about 1 g/day, about 2 g/day, about 5 g/day, about 10 g/day, about 15 g/day, about 20 g/day, about 25 g/day, about 30 g/day, about 35 g/day, about 40 g/day, about 45 g/day, about 50 g/day, about 55 g/day, or about 60 g/day. An effective amount of the galactomannans may be 1±0.1 g/day, 2±0.2 g/day, 5±0.5 g/day, 10±1 g/day, 15±1.5 g/day, 20±2.0 g/day, 25±2.5 g/day, 30±3.0 g/day, 35±3.5, g/day, 40±4.0 g/day, 45±4.5 g/day, 50±5.0 g/day, 55±5.5 g/day, or 60±6.0 g/day. An effective amount of the galactomannans may be 1 g/day, 2 g/day, 5 g/day, 10 g/day 15 g/day, 20 g/day, 25 g/day, 30 g/day 45 g/day, 50 g/day, 55 g/day, or 60 g/day.

### Methods of Treatment

The galactomannans of the present invention, or solid dosage forms or pharmaceutical compositions thereof, may serve as a useful therapeutic for SARS-CoV-2 infections or other viral infection as described herein. In particular, the galactomannans, may be useful in treating the symptoms of SARS-CoV-2 infection in a subject. In some embodiments, the subject is an adult (e.g., the subject is greater than 18 years old). In some embodiments, the subject is a child (e.g. the subject is less than 18 years old, less than 17 years old, less than 16 years old, less than 15 years old, less than 14 years old, less than 13 years old, less than 12 years old, less than 11 years old, less than 10 years old, less than 9 years old, less than 8 years old, less than 7 years old, less than 6 years old, less than 5 years old, less than 4 years old, less than 3 years old, less than 2 years old, less than 1 year old).

In some embodiments, the galactomannans are administered fewer than 48 hours following the diagnosis of a SARS-CoV-2 infection in the subject (e.g., fewer than 24 hours following the diagnosis of a SARS-CoV-2 infection in the subject, fewer than 12 hours following the diagnosis of a SARS-CoV-2 infection in the subject, fewer than 6 hours following the diagnosis of a SARS-CoV-2 infection in the subject, fewer than 3 hours following the diagnosis of a SARS-CoV-2 infection in the subject, at substantially the same time as a SARS-CoV-2 infection is diagnosed in the subject).

In some embodiments, the galactomannans are administered more than 30 minutes after the subject consumes food, e.g., more than 60 minutes, more than 90 minutes, or more than 120 minutes after the subject consumes food.

In some embodiments, including any of the foregoing embodiments, the galactomannans are administered to the subject at least once per day. In some embodiments, including any of the foregoing embodiments, the galactomannans are administered to the subject at least twice per day. In some embodiments, including any of the foregoing embodiments, the galactomannans are administered to the subject at least three times per day. In some embodiments, including any of the foregoing embodiments, the galactomannans are administered to the subject at least four times per day. In some embodiments, including any of the foregoing embodiments, the galactomannans are administered to the subject at least five times per day. In some embodiments, including any of the foregoing embodiments, the galactomannans are administered to the subject at least six times per day. In some embodiments, including any of the foregoing embodiments, the galactomannans are administered to the subject at least seven times per day. In some embodiments, including any of the foregoing embodiments, the galactomannans are administered to the subject at least eight times per day. In some embodiments, including any of the foregoing embodiments, the galactomannans are administered to the subject at least nine times per day. In some embodiments, including any of the foregoing embodiments, the galactomannans are administered to the subject at least ten times per day. In some embodiments, including any of the foregoing embodiments, the galactomannans are administered to the subject at least eleven times per day. In some embodiments, including any of the foregoing embodiments, the galactomannans are administered to the subject at least twelve times per day. In some embodiments, including any of the forgoing embodiments, the method comprises administering to the subject the galactomannans hourly, e.g., during waking hours.

### EXAMPLES

### Example 1: Purified Soluble Mannan Polysaccharide Use in Mild Cases of SARS-CoV-2: Pilot Feasibility Randomized, Open Label, Controlled Trial

### Design and Intervention

The study was a phase 3 pilot feasibility randomized, open Label, parallel arm-controlled trial conducted on 10 patients with mild COVID 19. This study was conducted on 10 patients identified between Sep. 15 and 19, 2020. Demographic data describing the 10 patients are summarized in Table 1.

TABLE 1
Demographic Data Describing Enrolled Patients and Laboratory Values

| Characteristics | Treated | Control |
|---|---|---|
| Number | 5 | 5 |
| Age | 28.4 ± 3.195 | 29.1 ± 4.53 |
| Gender (Female) | 4 | 4 |
| rRT PCR confirmed sars CoV-2 | 5 | 5 |
| infection | | |
| Time from symptom onset to randomization (median) | 1.80 days | 2 days |
| WHO CPS score (0-10) = 4 | Score 2 | Score 2 |
| 7 point ordinal scale | 3 | 3 |
| Co-existing conditions | Nil | Nil |

LABORATORY VALUES

| | | |
|---|---|---|
| CRP, mg/L | 1.13 ± 0.800.80 | 4.93 + 6.546.54 |
| D - Dimer, μg/L | 84.8 ± 78.2595 | 60 + 83.73 |
| Neutrophil count | 63 ± 15.245 | 3.82 + 9.19 |
| | 28.89 | 33.78 |
| Lymphocyte Count | ±10.69 | ±6.6 |
| | 0.691 ± 0.557 | 0.617 ± 0.17 |
| Lymphocyte to Monocyte Ration | 0.41 ± 0.91 | 0.38 ± 0.23 |
| RDW | 14.58 ± 1.9 | 16.69 ± 2.89 |
| Hemoglobin | 13.24 ± 0.2 | 12.27 ± 0.8 |
| ALT/SGPT | 19 ± 8.98 | 16 ± 5.33 |
| AST/SGOT | 29 ± 8.04 | 25.6 ± 3.43 |
| Creatinine | 0.66 ± 0.11 | 0.85 ± 0.26 |
| Ferritin | 27.96 ± 36.69 | 13.30 ± 7.47 |
| LDH | 219 ± 39.96 | 197 ± 3 6.63 |
| Cycle thresholds | N = 5 | N = 2 |
| Rd/Rp | 21.306 | 19.549 |
| Nucleocapsid gene | | |
| Envelope gene | 23.126 | 22.0895 |

The diagnosis and main criteria for inclusion included hospitalized and consenting participants between the ages of 18 and 45 with rRT-PCR positive for COVID-19, voluntarily and able to provide frequent nasopharyngeal and oropharyngeal swabs. Baseline screening was done with hematological and blood biochemistry tests for liver functions and kidney functions following which randomization was done using REDCap software into 2 study arms, the treatment arm receiving Prolectin-M and Standard of Care (SoC) and Control arm receiving SoC alone. All the 10 patients were hospitalized during drug administration of Prolectin-M for 5 days and followed-up for a whole period of 28 days.

Viral culture studies of SARS-CoV-2 indicate that date of onset of symptoms and cycle threshold levels relate to infectivity; a cycle threshold of f<25 is considered to be infectious. Most rRT-PCR approved kits, report Ct values for the RNA dependent RNA polymerase (Rd/Rp), envelope (E) protein and nucleocapsid (N) genes. All three genes are reported together for higher sensitivity. The droplet digital PCR measures expression of only nucleocapsid genes (N1 and N2) as an absolute number in copies/uL.

### Study Objective

The primary aim was to evaluate the efficacy, safety and feasibility of administering Prolectin-M versus the SoC for 5 days. The primary endpoint was a change in absolute count of Nucleocapsid gene and a rising Ct value, estimated from serial samples of RNA extracted from a nasopharyngeal swab. The swab was collected in all participants on days 1 (day of randomization), 3, 5, and 7. Clinical progression was estimated on a 7-point scale recorded on days 7, 21, and 28. A 2-point change was defined as clinical progression.

### 7-Point Severity Score (Ordinal Scale)

1 Not hospitalized, no limitations on activities.
2 Not hospitalized, limitation on activities.
3 Hospitalized, not requiring supplemental oxygen.
4 Hospitalized, requiring supplemental oxygen.
5 Hospitalized, on non-invasive ventilation or high flow oxygen devices.
6 Hospitalized, on invasive mechanical ventilation or extracorporeal membrane oxygenation (ECMO).
7 Death

### Overall Study Design and Plan: Description

The study was a single centric phase 3 pilot feasibility randomized, open Label, parallel arm-controlled trial conducted on 10 patients with mild COVID 19 disease. The Investigational drug was Prolectin-M, a Galectin antagonist. The objective of this trial was to study the feasibility of performing a definitive trial of using galectin antagonist-Prolectin-M as treatment for mild, symptomatic, rRT-PCR positive, COVID-19 patients.

Oral tablets of Prolectin-M were administered along with the best practice, SoC and compared against SoC alone. The intervention, Prolectin-M was administered as a multi dose regime of 4-gram tablets. Each tablet contained 2 grams of (1-6)-alpha-D-mannopyranosil (galactomannan) mixed with 2 grams of dietary fiber. Each participant took a single chewable tablet every hour, to a maximum of 10 hours in a day. Tablets were administered only during the daytime, for total of 5 days.

### Discussion of Study Design, Including the Choice of Control Groups

Study data were collected and managed using REDCap electronic data capture tools. REDCap (Research Electronic Data Capture) is a secure, web-based software platform designed to support data capture for research studies, providing 1) an intuitive interface for validated data capture; 2) audit trails for tracking data manipulation and export procedures; 3) automated export procedures for seamless data downloads to common statistical packages; and 4) procedures for data integration and interoperability with external sources.

### Selection of Study Population

### Inclusion Criteria

This single center trial enrolled hospitalized, eligible, and consenting participants between the ages of 18 and 45. All patients had to have an instrumental diagnosis for COVID-19; a positive rRT-PCR for SARS-CoV-2 obtained from an outpatient collection of nasopharyngeal swabs. Other inclusion criteria were the presence of symptoms that were not older than 72 hours and an ability to provide consent to undergo repeated collection of throat and nasal swabs over the 7-day period.

The diagnosis and main criteria for inclusion included hospitalized and consenting participants between the ages of 18 and 45 with rRT PCR positive for COVID-19, voluntarily and able to provide frequent nasopharyngeal and oropharyngeal swabs. Baseline screening was done with hematological and blood biochemistry tests for liver functions and kidney functions following which randomization was done using REDCap software into 2 study arms, the treatment arm receiving Prolectin-M and Standard of Care (SoC) and Control arm receiving SoC alone. All the 10 patients were hospitalized during drug administration of Prolectin-M for 5 days and followed-up for a whole period of 28 days.

### Exclusion Criteria

Exclusion criteria included oxygen saturation at admission≤96%, high temperature≥100 F (≥37.5° C.) not controlled on oral doses of acetaminophen, known history of diabetes on oral medications or insulin therapy or interleukin-6 levels≥3 times of laboratory reference range and/or significantly elevated levels of CRP, serum ferritin or d-dimer or a Lymphocyte/monocyte ratio≤3 or neutrophil/lymphocyte ratio≥5 or platelet count≤150,000 cells per microliter. Previously tested positive and recovered COVID-19 were excluded. Also participants on any chronic medications for more than 4 weeks before randomization or active malignancy or having any co-morbidity that increases risk of rapid disease progression were excluded.

### Removal of Patients from Therapy or Assessment

None of the 10 eligible recruited patients were not removed from therapy or assessment.

### Treatments

### Treatments Administered

After randomization, the treatment group was administered Prolectin-M orally in addition to the SoC drugs. Prolectin-M was administered orally once every hour up to a maximum dose through the day of 40 grams or 10 tablets a day. The intention was to mimic the viral replication cycle of 8-10 hours and also to ensure that the participant is consuming the tablets during the day under supervision of a research nurse. Each subject was encouraged to keep the tablet in their mouth for 1-2 minutes before it dissolved and swallowed. During a mealtime, breakfast, lunch, tea, and dinner, the subject had to wait for 30 minutes after the last meal before taking the next tablet. This was to avoid any potential drop in blood glucose as the tablets could block absorption of carbohydrates consumed in the meal. The control group patients continued with the Standard of Care treatment alone.

### Identity of Investigational Products

### Chemical Identification

Substance Name: Guar Gum
Chemical Classification category: Carbohydrates and derivatives
Catalog Name: Chewable tablets composed mainly of GRAS grade simple and complex carbohydrates.

### Composition

Main Ingredient: Mannans
Synonyms: Soluble mannans, polysaccharides purified from seeds of the plants Trigonella foenum-Graecum, Cyamopsis tetragonoloba, Acacia senegal, Acacia seyal and/or Ceratonia siliqua.
CAS #: 9000-30-0
EC #: 232-536-8
Other Ingredients: Food grade sorbitol, erythritol, malic acid, natural flavor and colors.

### Manufacturing/Use Information

Both finished products contained 3 g of mannans in a single dose (two chewable tablets or one succulent).

### Prior and Concomitant Therapy

No additional forms of therapy were administered prior to start of the trial and all forms of concomitant therapy were continued as per the protocol under supervision of the Investigator.

### Treatment Compliance

All 10 patients were hospitalized for the period from the day of randomization to Day 5 of receiving the doses as per the protocol and followed-up for a total of 28 days from the day of randomization on an outpatient basis if discharged. The medication was administered in the presence of a research nurse during daytime ensuring treatment compliance.

### Efficacy and Safety Variables

### Efficacy and Safety Measurements Assessed

A nasopharyngeal/oropharyngeal swab was transported to the research laboratory for RNA extraction in a viral transport media (#MG20VTM-3, MagGenome). All RNA extractions were carried out using the QIAamp Viral RNA mini kit (#52904, Qiagen) following the standard instructions as per the kit protocol.

For each blinded sample the Ct value for Rd/Rp and N, E gene, genes were reported. A sample was considered negative when no Ct value was determined, and no amplification curve was observed or if the Ct value was >29 for all three targets. Patients follow up was maintained for 28 days from the day of randomization.

### rRT PCR

The extracted RNA was analyzed on a rRT PCR platform: TRUPCR® SARS-CoV-2 RT qPCR KITV2 (#3B3043B Black Bio Biotech) SARS-CoV-2. To determine the efficiency of the PCR, Ct values obtained from a series of 5 template DNA dilutions of at least 3 different samples were graphed on the y-axis versus the log of the dilution on the x-axis. The Ct values assumed by the following equation were employed to calculate the logarithm of the recombinant gene copy numbers from: Ct=slope×log (Gene Copy Number)+1 where I in the formula acts as the intercept of standard curve.

Droplet Digital PCR (DDPCR): The RNA was also analyzed on the Droplet Digital PCR (BioRad, USA). This platform is FDA approved for emergency use authorization in COVID-19 [18]. The test is a partition-based endpoint single well RT-PCR test. The DDPCR was combined with rRT PCR because of its higher sensitivity and precision in low viral abundance samples and for the ability to provide absolute copy numbers and any resistance to inhibition often seen in rRT-PCR testing.

Demonstration of presence of IgG antibodies in the serum of all treated patients was done on Day 28.

The primary endpoint was a change in absolute count of Nucleocapsid gene and a rising Ct value, estimated from serial samples of RNA extracted from a nasopharyngeal swab. The swab was collected in all participants on days 1 (day of randomization), 3, 5, and 7. Clinical progression was estimated on a 7-point scale recorded on days 7, 21, and 28. A 2-point change was defined as clinical progression.

Adverse events were recorded from time of signature of informed consent and graded according to the Common Terminology Criteria for Adverse Events, version 5.0. Causality was assessed by the investigators for any serious adverse events.

### Primary Efficacy Variable(s)

Ct values, Estimation of copies/L of Nucleocapsid genes and Changes in ordinal score are the Primary Efficacy Variables taken under consideration in this trial.

### Statistical Methods Planned in the Protocol and Determination of Sample Size

A sample number of 10:5 in the test arm and 5 in the control arm were selected. Ct values and absolute copy numbers were compared using parametric, unpaired repeated t-test with Welch's correction or non-parametric Mann Whitney U test. A two-tailed, p<0.05, was considered statistically significant.

### Disposition of Patients

Participants were randomly assigned in a 1:1 ratio to receive a 4-gram tablet of Prolectin-M; a (1-6)-alpha-D-Mannopyranose, and SoC (Treatment group) or SoC (Control group) via a web-based secure centralized system (REDCap). An independent statistician provided a computer-generated assignment randomization list and blocked with varying block sizes unknown to the investigators. All patients in both treatment and control arms continued follow-up for the whole study period of 28 days. No withdrawals, premature terminations or deaths or serious adverse effects forcing discontinuation of treatment were observed.

### Protocol Deviations

There was one incidence of mild grade 1 diarrhea was observed in 1 patient receiving 10 tablets of Prolectin-M and the cause was identified as related to Prolectin-M and the dose was reduced to 5 tablets subsequently the patient recovered and continued the trial for the stipulated period.

### Data Sets Analyzed

Only one set of data including the 10 participants recruited for the trial was analyzed.

### Measurements of Treatment Compliance

The oral intervention was carried out only during daytime under the supervision of a qualified nurse to ensure that the patient consumed the tablet.

### Efficacy Results and Tabulations of Individual Patient Data

### Analysis of Efficacy

Efficacy was measured as a change in absolute count of Nucleocapsid gene and a rising Ct value, estimated from serial samples of RNA extracted from a nasopharyngeal swab. Efficacy data is summarized in Table 2.

TABLE 2
Summary of Efficacy Data

| | | | | | Difference |
|---|---|---|---|---|---|
| | | | | | between two |
| Characteristics | N | Treated | N | Control | groups |
| Change from baseline sars | 5 | 3/5 (60%) | 5 | No change | Yes |
| CoV-2 status day7 | | Negative | | | |
| Day 28-Serum Ig G (reactive) | 5 | All 5 reactive | 5 | 3 reactive; 2 non | Yes |
| | | | | reactive | |
| Reactive (≥1.00) | 5 | 9.08 (8.68, | 3 | 4.58 (0.00, | Yes |
| Serum IgG antibody titres on | | 10.1) | | 5.68) | |
| day 28 Median (IQR) | | | | | |
| Change in 7-point ordinal | 5 | All scored 1 | 5 | 3 scored 1; 2 scored | Yes |
| scale day 14 | | | | 2; restricted as they | |
| | | | | were positive | |
| Change in 7-point ordinal | 5 | No change from | 5 | Yes: 2 to 1 | No |
| scale day 21 | | last assessment | | | |
| Change in 7-point ordinal | 5 | No change from | 5 | No change from last | No |
| scale day 28 | | last assessment | | assessment | |
| Cycle threshold | 5 | 25.86 | 5 | 9.446 | Yes 16.41 |
| on day 7 - mean | | | | | 95% CI, 0.3527 to |
| increase from | | | | | 32.48, p = 0.047 |
| baseline Rd/Rp + | | | | | |
| Nucleocapsid gene | | | | | |
| Envelope gene | 5 | 28.27 | 5 | 10.52 | Yes, 17.7595% |
| | | | | | CI, 0.1321 to |
| | | | | | 35.63, p = 0.05 |

### Tabulation of Individual Response Data

TABLE 3
Summary of Adverse Events

| Mild | | | Moderate | | Severe | | Total | | Total |
|---|---|---|---|---|---|---|---|---|---|
| Related | | NR | Related | NR | Related | NR | Related | NR | R + NR |
| Gastrointestinal | | | | | | | | | |
| System | | | | | | | | | |
| Event 1 | 1 (20%) | | | | | | | | |
| Diarrhea | | | | | | | | | |

### Results

By day 7, following treatment with Prolectin-M, Ct value of Rd/Rp+N gene increased by 16.41 points, 95% (CI 0.3527 to 32.48, p=0.047). Similarly, Ct values of the small envelope (E) gene also increased by 17.75 points (95% CI, −0.1321 to 35.63, p=0.05). The expression of N1, N2 genes went below detectable thresholds by day 3 (Mann Whitney U=0.000, p<0.029).

rRT-PCR testing done in the clinic on day 1, 7, and 14 had 3 participants (60%) turn negative by day 7 and all turned negative by day 14 and stayed negative until day 28. In the SoC group 2 participants had zero detectable viral loads at baseline, 2 participants tested negative on day 14, and the last participant remained positive on day 28. There were no serious adverse events, and all participants were clinically asymptomatic before day 28 with reactive immunoglobulin G (IgG).

### Discussion and Overall Conclusion

This was the first ever reported randomized controlled trial of a galectin antagonist against SARS-CoV-2. Blocking N terminal domain (NTD) of S1 subunit, present on spike protein, was seen to have significantly lowered viral gene expression. All five participants in treatment group demonstrated a rapid drop by day 3 in copies/μL of Nucleocapsid protein gene.

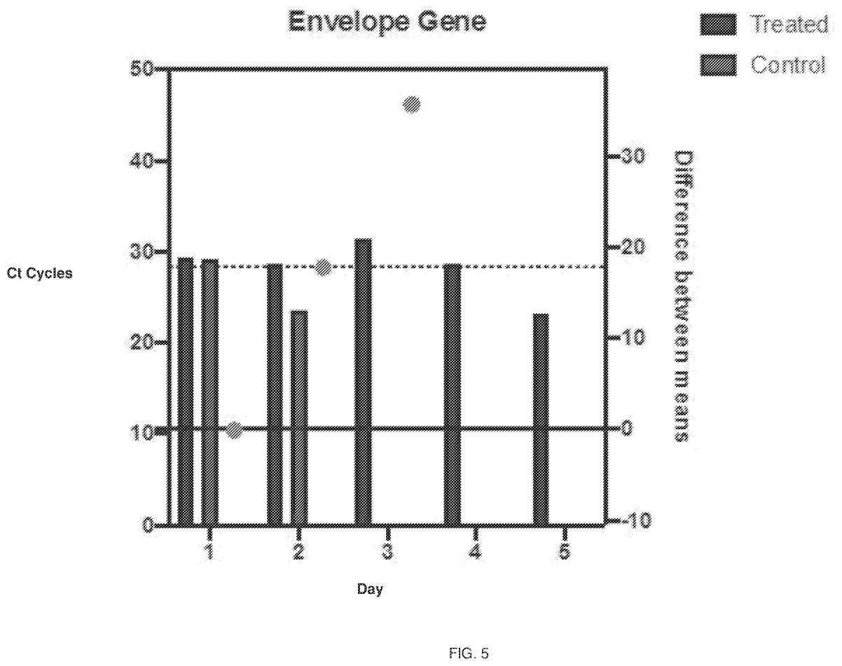

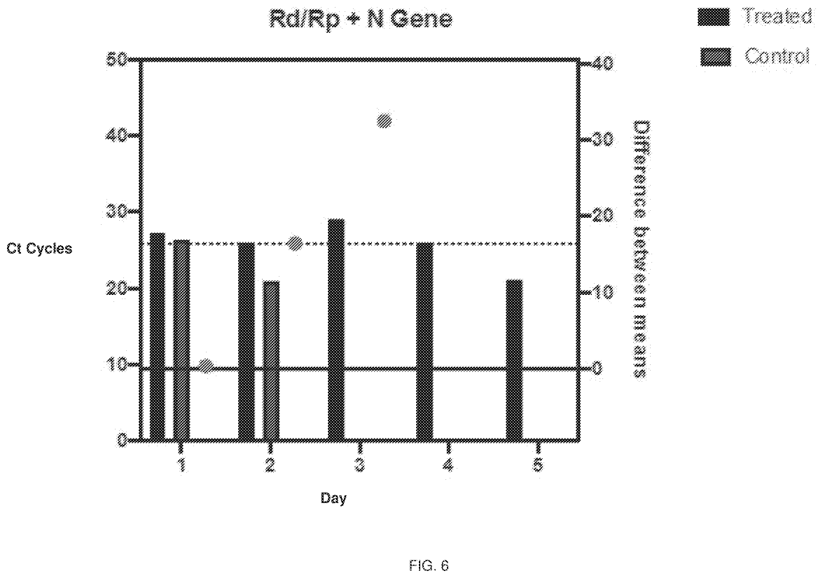

A higher Ct value correlates with lower risk of infectiousness, as reported from a cohort in England between January to May 2020. There was rise in Ct>29, for genes, Rd/Rp, N and E genes in the treated group (FIG. 5, FIG. 6). However, participants in control group (001 and 007), remained potentially infective even on day 7 and day 14.

The detection of anti-spike protein IgG on day 28, was an expression of humoral response against the virus. The N protein is highly immunogenic compared to the S protein and in the absence of glycosylation sites on it results in N-specific neutralizing antibody production early in the stage of acute infection. All participants in the active arm of the trial had a reactive IgG. This trial achieved its primary objective of demonstrating the effect of Prolectin-M on lowering viral infectiousness. Strengths of this study were randomization, concealed allocation, and one hundred percent compliance to treatment, and all procedures in the protocol. There was no incidence of any serious adverse events. A higher cut off value for either cycle threshold or copy numbers on ddPCR as an eligibility criterion could have demonstrated a more significant treatment effect. Hence future trials will need to use either a pre-determined lower (<16) baseline Ct value or a higher copy number for ddPCR.

Galectin antagonists can potentially prevent SARS-CoV-2 entry into human cells. A larger clinical trial will give us the evidence needed to understand its true clinical benefit. Due to the excellent safety profile of galectin antagonists, additional methods of delivery, such as an intravenous, or subcutaneous injection should be examined for a reduction in the number of systemically expressed copies of the virus. In addition, Prolectin-M could be very effective as a prophylactic to stop the community spread of the disease. The results from this pilot and feasibility trial sets the stage for a definitive large randomized controlled clinical trial using galactomannans. as Galectin antagonists have a potential role as a Post Infection Immunization (PII) Lowering the basic Reproduction number (R0) can reduce SARS-CoV-2 contagiousness and transmissibility, while protecting the individual from serious COVID-19.

### Example 2: Antiviral Testing-SARS-CoV-2

### Test(s) Performed

In-vitro assay to demonstrate inhibition of a patient sample derived strain of SARS-CoV-2 virus infection of Vero (African green monkey kidney cells, Vero (ATTC® CCL-81™))

### Objective(s) of the Test(s)

To test the viral inhibition effect of the given compounds (blinded) ProLectin-M. 72 hours after infection. The end point was % viral reduction. Viral reduction was defined as:

$$\% Viral Reduction = \frac{\begin{matrix}number of viral particles in infection control - \\ number of viral particles exposed\end{matrix}}{number of viral particles in infection control}$$

### A Brief Description of the Test Methods, Including Sample Size, Device(s) Tested, and Any Consensus Standard(s) Utilized

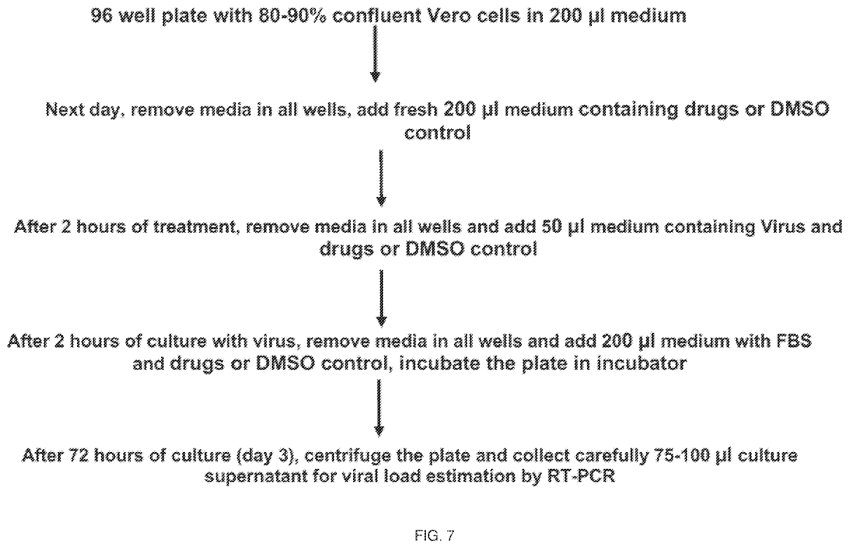

All petri dishes, dilution tube racks, and host-containing apparatus will be labeled with the following information: virus, host, test agent. Briefly, a flask of Vero cells grown in cell culture media containing 10% fetal bovine serum (FBS) was used. Cells were seeded in 96-well plate one day before experiment, next day medium is removed, cells are treated with test compound for 2 hours in 200 μl medium. After 2 h, medium is removed and then cells were infected with SARS-CoV-2 at a multiplicity of infection (MOI) of 0.01 in the presence of same concentrations of test compound or PBS control. The dose-response curves are determined by quantification of viral RNA copy numbers in the supernatant of infected cell at 72 h post infection (p.i.). A summary of the experimental workflow is provided in FIG. 7.

The test compounds are as given below
ProLectin-M (1750 mg). It is a blend of three compounds:
1. G-01: 23.55% w/w (MW: 20,000-40,000 daltons)
2. G-02: 46.66% w/w (MW: 20,000-40,000 daltons)
3. G-03: 17.56% (MW: 663.4 g/mol or 0.53M per tablet)
   The above three were blended to form the tablet.—Prolectin-M and administered in humans as 1 tablet every hour for 8 hours in a day for 14 days.

### Acceptance Criteria

The experiment was done in duplicates and the values were averaged to calculate % viral reduction. The regression equation for viral particles Vs Ct value of the N-gene specific to SARS-CoV-2 virus (y=3.5422x+4.786, R²=0.99) (X=Number of viral particles, y=ctValue) Number of particles are calculated using, X=(40.786−Ct_{Rd/Rp-genes@different time points})/3.5422. Any log reduction in the % viral particles in the experiments is considered to an efficacy of the test compounds to block viral infectivity to the vero cells. Viral reduction was calculated using the following formula:

$$\% of viral reduction = \frac{\begin{matrix}( number of viral particles in infection control - \\ number of viral particles exposed to test drugs )\end{matrix}}{Number of viral particles in infection control}$$

### Results Summary

Table 4 below gives a summary of the results of the experiments. Protocol 1 and 2 provided nearly 2 log reduction in viral copy numbers when compared to control. Treating vero cells with drug before infecting with virus (Protocol 1) and culturing the vero cells with virus and later treating with the test drugs demonstrated a near 2 log reduction (99% is a 2 log reduction) in viral copy numbers.

TABLE 4
Summary of Results.

| | | | Test compounds |
|---|---|---|---|
| | | Range | compared to |
| | | of Dose | Control |
| Test | | concen- | (% of viral |
| Compound | Experiment | tration | load reduction) |
| Prolectin-M | Protocol 1 - Vero cells | 6.25 μg/ml, | 98%, |
| | in plate treated with | 12.50 μg/ml, | 95%, |
| | SARS-Cov-2 virus and | 25 μg/ml, | 94%, |
| | test drugs/control | 50 μg/ml, | 93%, |
| | (DMSO) | 100 μg/ml, | 95%, |
| | | 200 μg/ml | 84%, |
| Prolectin-M | Protocol 2 - Vero cells | 6.25 μg/ml, | 96%, |
| | cultured with SARS- | 12.50 μg/ml, | 94%, |
| | CoV-2 virus followed by | 25 μg/ml, | 95%, |
| | treatment with medium | 50 μg/ml, | 94%, |
| | spiked with drugs/ | 100 μg/ml, | 92%, |
| | control (Fetal Bovine | 200 μg/ml | 86%, |
| | Serum) | | |

### Protocol 1

TABLE 5
Assay details:

| Assay | | |
|---|---|---|
| Performed | Materials Used | Manufacturer |
| Viral RNA | MaxMAX ™ Viral/Pathogen II | Applied Biosystems (by |
| Extraction | Nucleic Acid Isolation Kit | Thermo Fisher Scientific |
| qRT-PCR | Q-line ® Molecular nCOV19 | POCT Services Private |
| | RT-PCR Detection Kit | Limited |
| | (Multiplex) | |

Tested Concentrations (μg/mL) 6.25, 12.5, 25, 50, 100, 200.

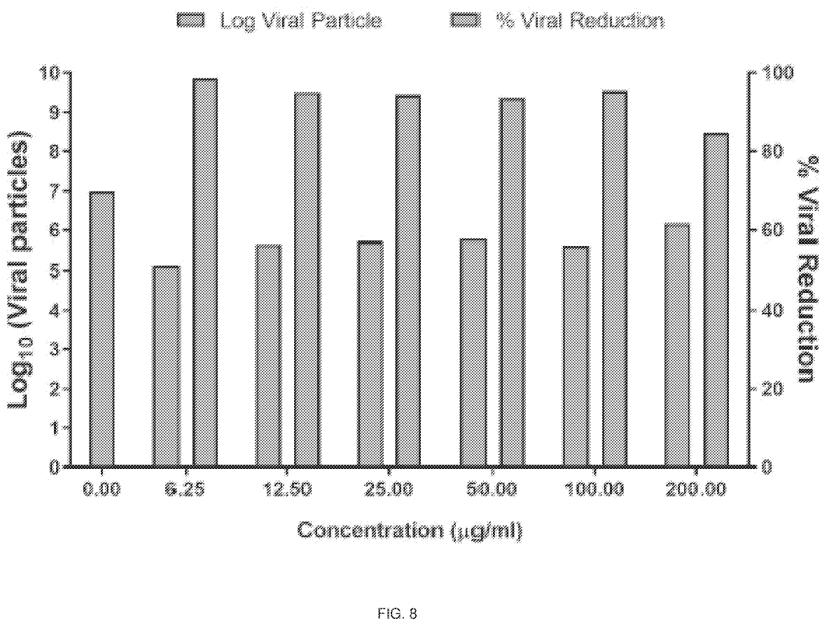

The Prolectin-M showed 98, 95, 94, 93, 95, and 84% viral reduction from 6.25 μg/mL until 200 μg/mL. The viral particles reduced from 10^{6.9} to 10^{5.1}. The experiment was done in duplicates and the values were averaged to calculate % viral reduction. The regression equation for particles The regression equation for viral particles Vs Ct value of the N-gene specific to SARS-CoV-2 virus (y=−3.5422x+40.786, R²=0.99) (X=Number of viral particles, y=Ct value). Number of viral particles are calculated using X=(40.786−Ct_{Rdrp-gene@different time points})/3.5422 (FIG. 8).

$$\% Viral reduction = \frac{\begin{matrix}number of viral particles in infection control - \\ number of viral particle exposed to drug ( test )\end{matrix}}{number of viral particles in infection control} \times 100$$

### Protocol 2

TABLE 6
Assay details:

| Assay | | |
|---|---|---|
| Performed | Materials Used | Manufacturer |
| Viral RNA | MaxMAX ™ Viral/Pathogen II | Applied Biosystems (by |
| Extraction | Nucleic Acid Isolation Kit | Thermo Fisher Scientific |
| qRT-PCR | Q-line ® Molecular nCOV19 | POCT Services Private |
| | RT-PCR Detection Kit | Limited |
| | (Multiplex) | |

Tested Concentrations (μg/mL) 6.25, 12.5, 25, 50, 100, 200.

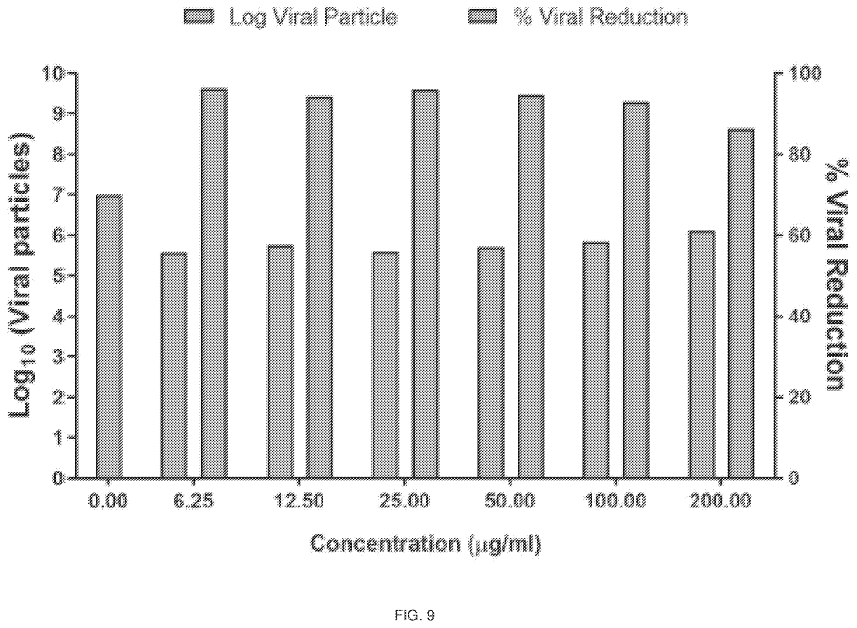

The Prolectin-M showed 96, 74, 95, 94, 92, and 86% viral reduction from 6.25 μg/mL to 200 μg/mL. The viral particles reduced from 10^{6.9} to 10^{5.5}. The experiment was done in duplicate and the values were average to calculate % viral reduction. The regression equation for viral particles Vs Ct value of the N-gene specific to SARS-CoV-2 virus (y=−3.5422x+4.786, R²=0.99) (X=Number of viral particles, y=Ct value). Number of viral particles are calculated using X=(40.786−Ct_{Rdrp-gene @ different time points})/3.5422 (FIG. 9).

$$\% Viral reduction = \frac{\begin{matrix}number of viral particles in infection control - \\ number of viral particles exposed to drug ( test )\end{matrix}}{number of viral particles in infection control} \times 100$$

### Example 3: NMR analysis of Prolectin-M

### Glycosyl Linkage Analysis

A Prolectin-M tablet was suspended in 50 mL of nanopure water. The supernatant of the suspension was lyophilized. Aliquots of 1.0 mg of Prolectin-M were used for linkage analysis. The sample was stirred in 400 μL of anhydrous dimethyl sulfoxide (DMSO) for 2 days until the samples were dissolved. Permethylation was achieved by two rounds of treatment with sodium hydroxide (NaOH) base (30 min) and iodomethane (90 min). The sodium hydroxide base was prepared according to the protocol described by Anumula and Taylor (1992) Anal. Biochem. 203:101-108. Briefly, to 100 μL of 50% w/w NaOH was added 200 μL of methanol (MeOH), and the mixture was vortexed. Then 2 mL of DMSO was added, and the base solution was vortexed and centrifuged repeatedly up to 5 times to remove residual sodium carbonate. After the final extraction, 2 mL of DMSO was added to the NaOH pellet and the solution was vortexed. Of this final base solution, 400 μL was added to the sample, and the mixture was sonicated for 30 min. Then, 100 μL of iodomethane was added, and the sample was stirred magnetically at room temperature for 45 min. A second round of base and then iodomethane was performed to ensure complete methylation.

The permethylated materials were hydrolyzed with 2 M TFA for 2 h at 121° C. and dried down with isopropanol under a stream of nitrogen. The samples were then reduced with 10 mg/mL NaBD₄ in 100 mM NH₄OH overnight, neutralized with glacial acetic acid, and dried with methanol. Finally, the sample was O-acetylated using 250 μL of acetic anhydride and 250 μL of concentrated trifluoroacetic acid (TFA) at 45° C. The sample was dried under N₂ stream, reconstituted in dichloromethane, and washed with nanopure water before injection into GC-MS (Table 7).

The resulting partially methylated alditol acetates (PMAAs) were analyzed on an Agilent 7890A GC interfaced to a 5975C MSD; separation was performed on a Supelco 2331 fused silica capillary column (30 m×0.25 mm ID) with a temperature gradient detailed in Table 1. The method was a derivation of the linkage method detailed by Heiss et al. (2009) Carbohydr. Res. 344: 915-920.

TABLE 7

| | Rate (° C./min) | Value (° C.) | Hold Time (min) |
|---|---|---|---|
| Initial | | 60 | 1 |
| Ramp1 | 27.5 | 170 | 0 |
| Ramp2 | 4 | 235 | 2 |
| Ramp3 | 3 | 240 | 12 |

### NMR Analysis

Sample Prolectin-M 9.2 mg was weighed and resuspended in 600 μl of D₂O. The supernatant was transferred into an NMR tube. Ten microliters of 1 mM DSS was added into the NMR tube as internal standard. The sample was analyzed at 34° C. (to shift the water signal away from the anomeric signals of Man) with a Bruker 900 MHz NMR instrument equipped with a cryoprobe. A standard zgf2pr pulse sequence was employed with a pre-saturation sequence for water suppression. Pulse sequences, hsqcetgpsisp2, clmlevphpr, and noesygpphf2pr, were applied for collecting HSQC, TOCSY, and NOESY spectrum, respectively.

### Results

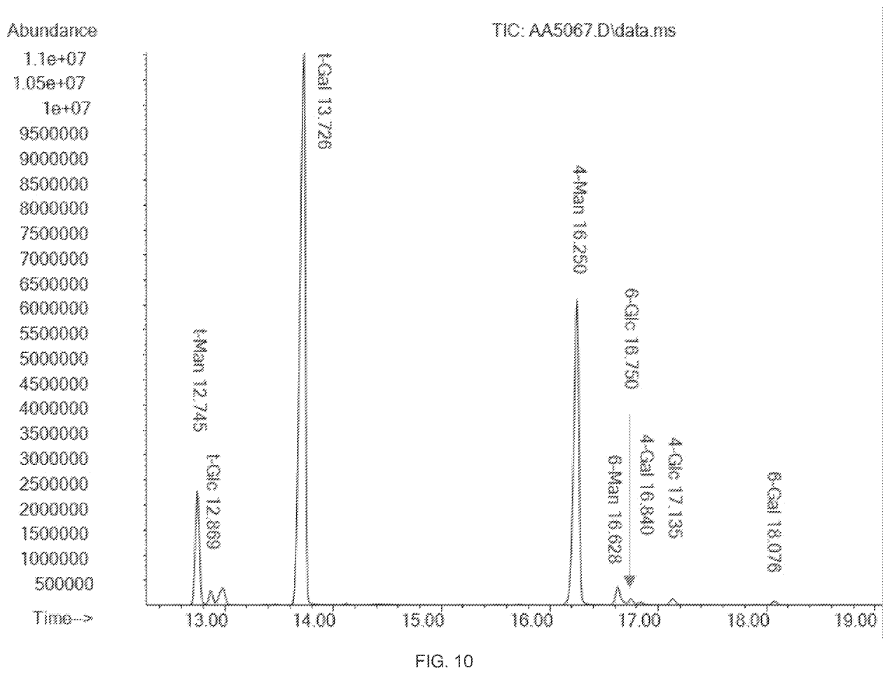

The glycosyl linkage analysis chromatogram is shown in FIG. 10, and the results are listed in Table 8. In summary, the most abundant linkages of Prolectin-M were 4-Manp, 4,6-Manp, and t-Galp, suggesting a galactomannan structure. The ratio of t-Galp to Manp of the sample was about equal to the ratio of branching Manp to all Manp, or about 2:3. This indicated that the mannan backbone is glycosylated with about 2 Gal on every 3 Man residues. The ratio of terminal Manp to internal Manp was about 1:10, suggesting an average back bone chain length of 11 Man residues. Together with the side chain Galp residues, this resulted in an average molecular weight of about 3 kDa.

TABLE 8
Relative percentage of each detected linkage in Prolectin-M.

| | Relative Area |
|---|---|
| Glycosyl Linkage Residue | Percentage (%) |
| Terminal Mannopyranosyl Residue (t-Manp) | 5.7 |
| Terminal Glucopyranosyl Residue (t-Glcp) | 0.8 |
| Terminal Galactopyranosyl Residue (t-Galp) | 36.5 |
| 4-linked Mannopyranosyl Residue (4-Manp) | 19.4 |
| 6-linked Mannopyranosyl Residue (6-Manp) | 1.1 |
| 6-linked Glucopyranosyl Residue (6-Glcp) | 0.4 |
| 4-linked Galactopyranosyl Residue (4-Galp) | 0.2 |
| 4-linked Glucopyranosyl Residue (4-Glcp) | 0.4 |
| 6-linked Galactopyranosyl Residue (6-Galp) | 0.3 |
| 4,6-linked Mannopyranosyl Residue (4,6-Manp) | 35.0 |
| 4,6-linked Glucopyranosyl Residue (4,6-Glcp) | 0.3 |
| Total | 100.0 |

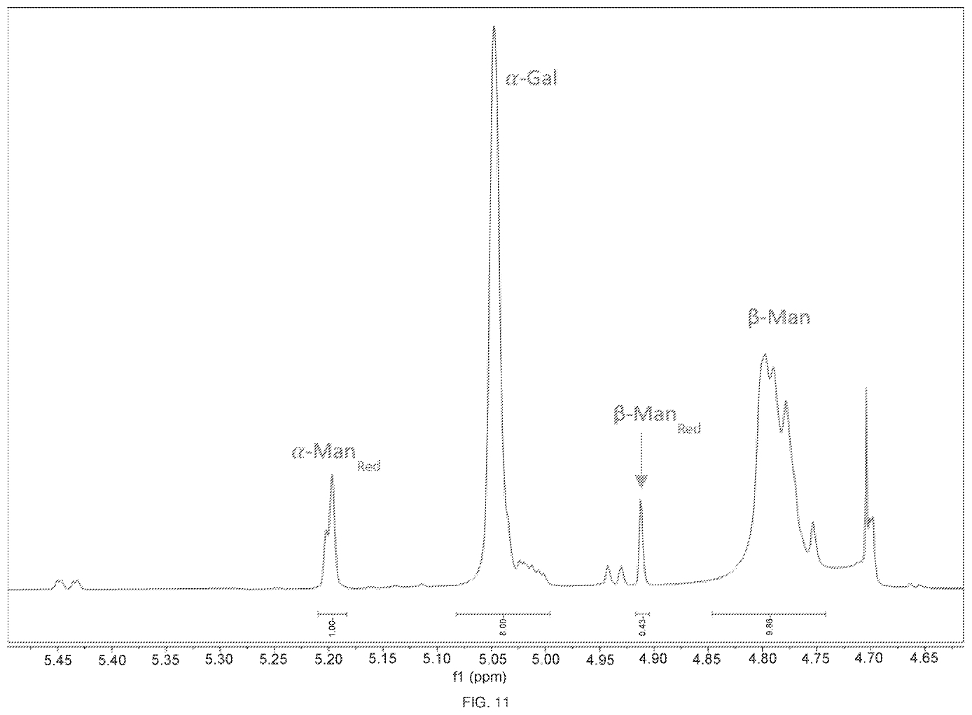

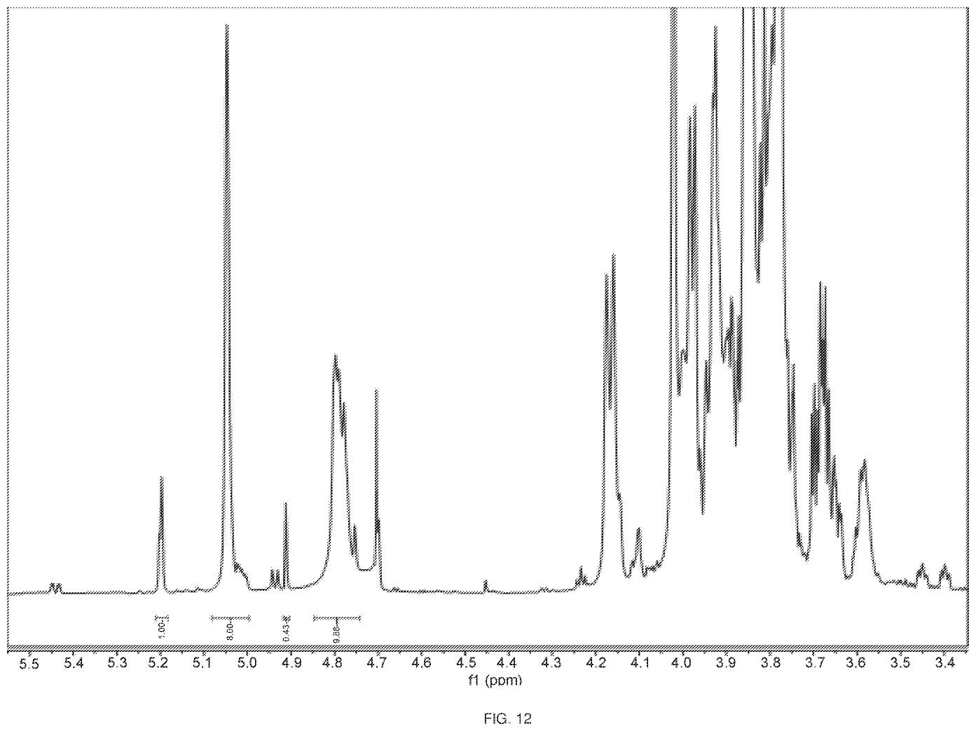

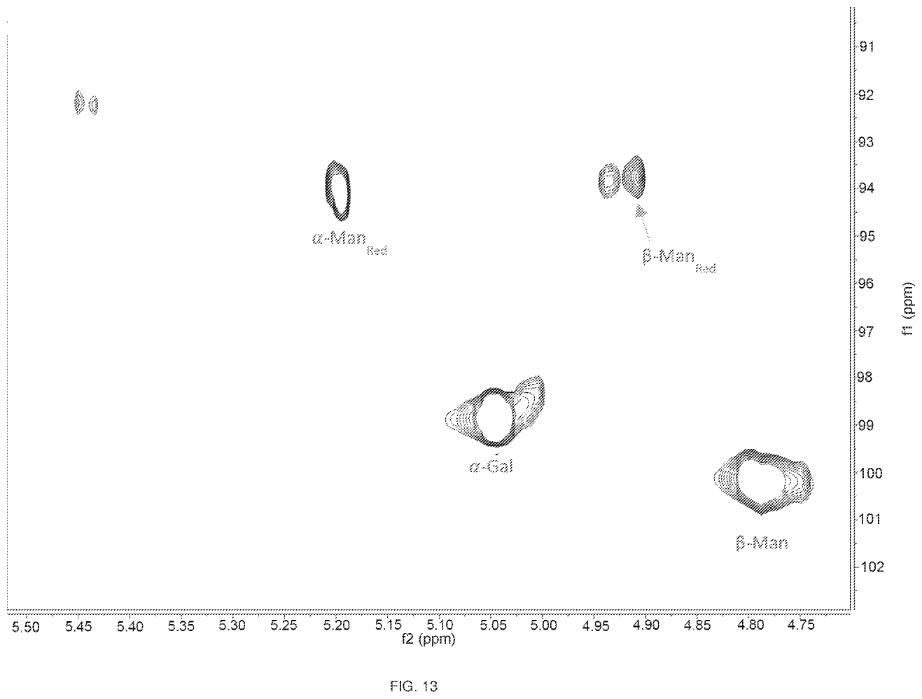

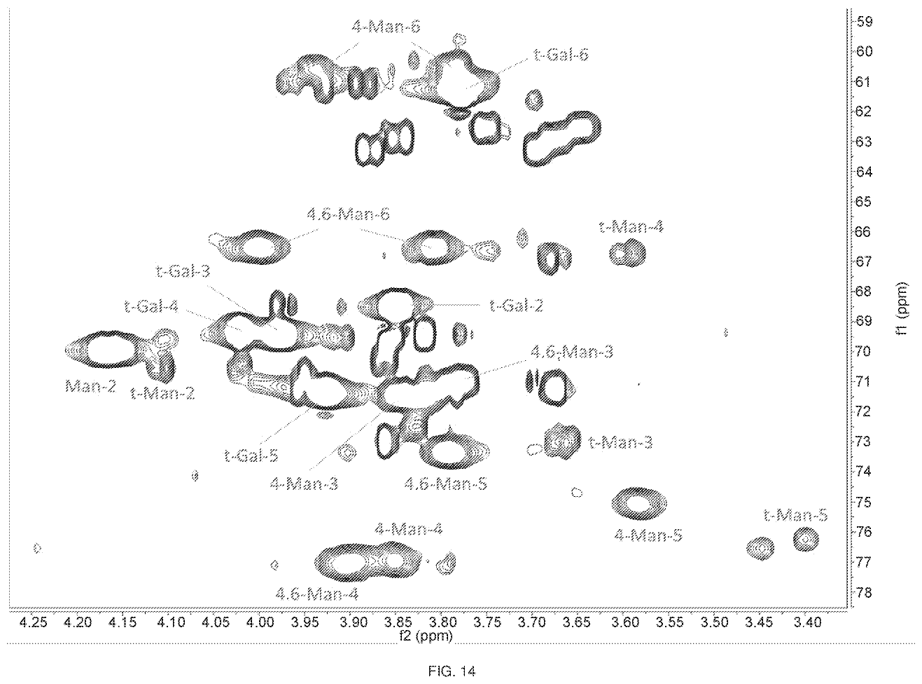

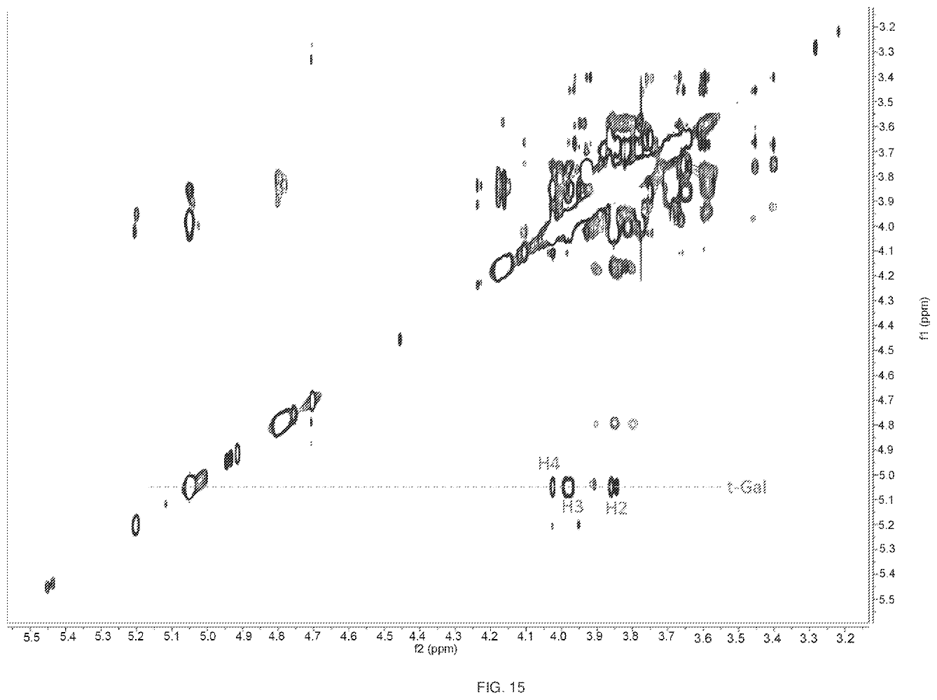

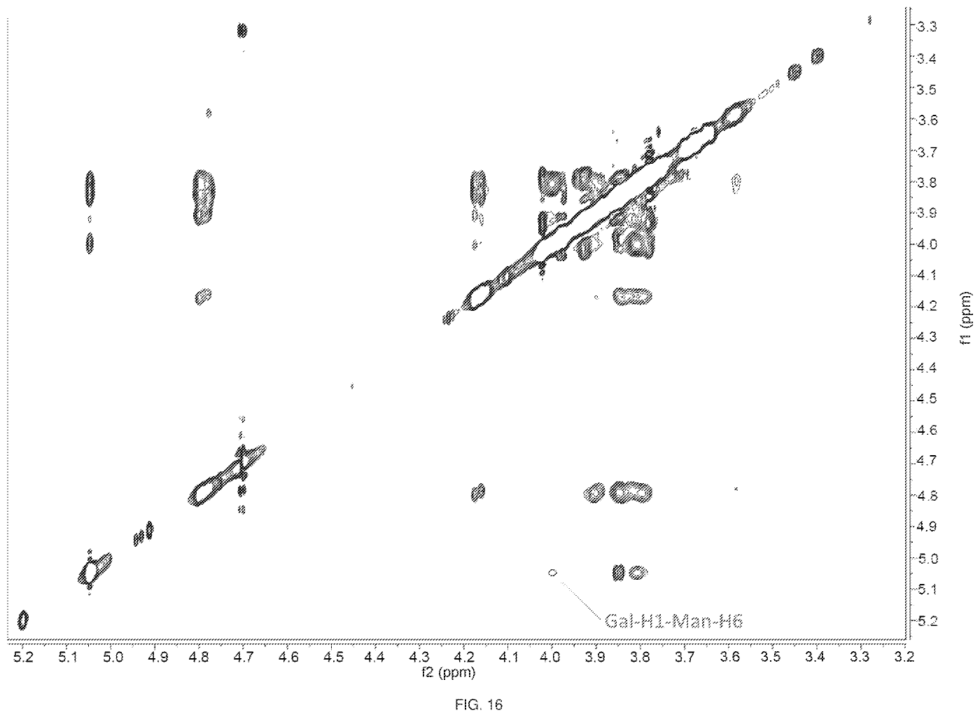

The NMR spectra are shown in FIGS. 11-16. The integration ratio between α-Gal H1 (8.00) and Man H1 [including reducing end Man (1.43 for both α and β configurations) and inner Man (9.86)] in the ¹H-NMR spectrum was about 1:1.4, suggesting the mannan backbone is glycosylated with about 2 Gal on every 3 Man residues, in agreement with the results from the glycosyl linkage analysis. The ¹H/¹³C cross-peaks are assigned and labeled in FIG. 13 and FIG. 14. The chemical shifts of these major glycosyl residues are summarized in Table 8. The assignment of a-Gal signals was based on a TOCSY spectrum (FIG. 15), while the assignment of backbone Man cross-peaks was based on literature (see references below). A NOESY spectrum confirmed the linkage between t-Gal and backbone Man by NOEs between α-Gal H1 and Man H6 (FIG. 16). Therefore, the NMR results support the linkage analysis data, both confirmed the major component of Prolectin-M is galactomannan.

TABLE 8
Chemical shift assignment of major glycosyl residues of Prolectin-M.

| Glycosyl | C/H-1 | C/H-2 | C/H-3 | C/H-4 | C/H-5 | C/H-6 |
|---|---|---|---|---|---|---|
| α-Man_{Red} | 94.06/5.2 | 71.06/4.0 | 72.99/3.9 | 77.11/3.8 | — | — |
| β-Man_{Red} | 93.76/4.9 | 68.50/4.0 | — | — | — | — |
| t-α-Gal | 98.88/5.0 | 68.48/3.9 | 69.45/4.0 | 69.35/4.0 | 71.37/3.9 | 61.31/3.8 |
| 4-β-Man | 100.20/4.8 | 69.99/4.2 | 71.53/3.8 | 76.96/3.9 | 75.11/3.6 | 60.60/3.9, 3.8 |
| 4,6-β-Man | 100.20/4.8 | 69.99/4.2 | 70.99/3.8 | 77.10/3.9 | 73.34/3.8 | 66.53/4.0, 3.8 |
| t-β-Man | 100.20/4.8 | 70.57/4.1 | 73.08/3.7 | 66.71/3.6 | 76.26/3.4 | 62.92/3.9, 3.8 |

Prolectin-M used in NMR analysis was the supernatant or easily-soluble portion of Prolectin-M, i.e., galactomannan molecules with lower molecular sizes. This agreed with the results from the linkage analysis and with the observation in the 1D ¹H-NMR spectrum of relatively large peaks for the reducing end Man residues.

Other embodiments are in the claims.

1. A method of treating a SARS-CoV-2 infection in a subject, comprising administering to the subject an effective amount of galactomannans.

2. The method of claim 1, wherein administration of the effective amount of galactomannans to the subject reduces the SARS-CoV-2 infectivity of the subject.

3. The method of claim 1, wherein administration of the effective amount of galactomannans decreases the copy number of SARS-CoV-2 RNA polynucleotides present in a biological sample from the subject.

4. (canceled)

5. The method of claim 1, wherein administration of the effective amount of galactomannans increases the copy number of a SARS-CoV-2 gene.

6. The method of claim 5, wherein the SARS-CoV-2 gene is the SARS-CoV-2 envelope protein gene or the SARS-CoV-2 nucleocapsid gene or the SARS-CoV-2 RNA-dependent RNA polymerase gene.

7. (canceled)

8. (canceled)

9. The method of claim 5, wherein the copy number of the SARS-CoV-2 gene is measured in a real time polymerase chain reaction experiment.

10. The method of claim 5, wherein the copy number of the SARS-CoV-2 gene is measured using a biological sample obtained from the subject.

11. (canceled)

12. The method of claim 1, wherein administration of the effective amount of galactomannans to a subject results in an increase in the Immunoglobulin G antibody titer in the subject.

13. The method of claim 1, wherein the galactomannans are formulated in a pharmaceutical composition with a pharmaceutically acceptable carrier.

14. The method of claim 13, wherein the pharmaceutical composition is a solid oral dosage form.

15. The method of claim 14, wherein the solid oral dosage form is a chewable tablet.

16. The method of claim 1, wherein the galactomannans, or a pharmaceutical composition thereof, are administered to the subject at least one time a day.

17. The method of claim 16, wherein the galactomannans, or a pharmaceutical composition thereof, are administered to the subject at least two times a day.

18. The method of claim 16, wherein the galactomannans, or pharmaceutical composition thereof, are administered to the subject up to ten times a day.

19. The method of claim 17, wherein the galactomannans, or a pharmaceutical composition thereof, are administered to the subject one time per hour during each hour that the subject is awake.

20. The method of claim 1, wherein the subject holds the galactomannans, or a pharmaceutical composition thereof, in their mouth for at least one minute before swallowing.

21. (canceled)

22. The method of claim 1, wherein the subject holds the galactomannans, or a pharmaceutical composition thereof, in their mouth for at least five minutes before swallowing.

23. The method of claim 1, wherein the galactomannans, or a pharmaceutical composition thereof, is administered at least thirty minutes after the subject has last eaten.

24. A method of reducing the number of SARS-CoV-2 virons in a sample of cells, comprising contacting the cell with an effective amount of galactomannans.

25. The method of claim 24, wherein the number of virons in the sample of cells is reduced by at least 50%.

26. (canceled)

27. (canceled)

28. (canceled)

29. (canceled)
